(19) United States
(12) Patent Application Publication
(10) Pub. No.: US 2025/0149117 A1
(43) Pub. Date: May 8, 2025

(54) TECHNIQUES FOR DETECTING DE NOVO AND RARE VARIANTS USING A FAMILY GRAPH REFERENCE

(71) Applicant: Seven Bridges Genomics Inc., Charlestown, MA (US)

(72) Inventors: Özem Kalay, Istanbul (TR); Sinem Budak, Ankara (TR); Deniz Turgut, Istanbul (TR); Amit Jain, London (GB)

(21) Appl. No.: 18/504,929

(22) Filed: Nov. 8, 2023

(51) Int. Cl. G16B 30/10; G16B 30/20; G16B 40/20

(52) U.S. Cl. CPC G16B 30/10; G16B 30/20; G16B 40/20

(57) ABSTRACT

Described herein are techniques for genotyping a family trio by constructing a family genomic reference graph and analyzing sequence reads from members of the family trio using the family genomic reference graph, the family trio comprising a child and biological parents of the child. In some embodiments, the techniques include obtaining the sequence reads; aligning the sequence reads to an initial genomic reference; identifying, based on results of the aligning, an initial plurality of variants; generating the family genomic reference graph using the initial plurality of variants; aligning at least some of the sequence reads to the family genomic reference graph; and identifying, based on results of aligning the at least some of the sequence reads to the family genomic reference graph, an updated plurality of variants comprising a respective updated set of variants for each of the members of the family trio.

### BACKGROUND

Rare diseases are commonly caused by the presence of germline mutations in the patients' genome. Germline mutations are either acquired from the genomes of the biological parents following the rules of Mendelian inheritance, or acquired de novo, due to errors introduced during the process of reproduction (i.e., germline de novo variants). While germline de novo variants are rare, they have been shown to be a major cause of severe early-onset genetic disorders such as intellectual disability, autism spectrum disorder, and other developmental diseases.

### SUMMARY

Some aspects provide for a method for genotyping a family trio by constructing a family genomic reference graph and analyzing sequence reads from each member of the family trio using the family genomic reference graph, the family trio comprising a child and biological parents of the child, the method comprising: using at least one computer hardware processor to perform: obtaining the sequence reads, the sequence reads having been previously obtained by sequencing biological samples obtained from the members of the family trio; aligning the sequence reads to an initial genomic reference using at least one data structure representing the initial genomic reference; identifying, based on results of the aligning, an initial plurality of variants comprising a respective initial set of variants for each of the members of the family trio; generating the family genomic reference graph using the initial plurality of variants, the family genomic reference graph comprising nodes and edges connecting the nodes, the generating comprising generating at least one data structure storing data specifying the nodes and the edges; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, an updated plurality of variants comprising a respective updated set of variants for each of the members of the family trio.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method for genotyping a family trio by constructing a family genomic reference graph and analyzing sequence reads from each member of the family trio using the family genomic reference graph, the family trio comprising a child and biological parents of the child, the method comprising: using at least one computer hardware processor to perform: obtaining the sequence reads, the sequence reads having been previously obtained by sequencing biological samples obtained from the members of the family trio; aligning the sequence reads to an initial genomic reference using at least one data structure representing the initial genomic reference; identifying, based on results of the aligning, an initial plurality of variants comprising a respective initial set of variants for each of the members of the family trio; generating the family genomic reference graph using the initial plurality of variants, the family genomic reference graph comprising nodes and edges connecting the nodes, the generating comprising generating at least one data structure storing data specifying the nodes and the edges; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, an updated plurality of variants comprising a respective updated set of variants for each of the members of the family trio.

Some aspects provide for at least one non-transitory computer-readable storage medium storing processor-executable instructions that, when executed by at least one computer hardware processor, causes the at least one computer hardware processor to perform a method for genotyping a family trio by constructing a family genomic reference graph and analyzing sequence reads from each member of the family trio using the family genomic reference graph, the family trio comprising a child and biological parents of the child, the method comprising: using at least one computer hardware processor to perform: obtaining the sequence reads, the sequence reads having been previously obtained by sequencing biological samples obtained from the members of the family trio; aligning the sequence reads to an initial genomic reference using at least one data structure representing the initial genomic reference; identifying, based on results of the aligning, an initial plurality of variants comprising a respective initial set of variants for each of the members of the family trio; generating the family genomic reference graph using the initial plurality of variants, the family genomic reference graph comprising nodes and edges connecting the nodes, the generating comprising generating at least one data structure storing data specifying the nodes and the edges; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, an updated plurality of variants comprising a respective updated set of variants for each of the members of the family trio.

Some aspects provide for a method for genotyping a family trio by using a family genomic reference graph and analyzing sequence reads from each member of the family trio using the family genomic reference graph, the family trio comprising a child and biological parents of the child, the method comprising: obtaining the sequence reads, the sequence reads having been previously obtained by sequencing biological samples obtained from each of the members of the family trio; obtaining at least one data structure storing data specifying nodes and edges of a family genomic reference graph, the family genomic reference graph having been previously generated; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, a set of variants for each of the members of the family trio.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method for genotyping a family trio by using a family genomic reference graph and analyzing sequence reads from each member of the family trio using the family genomic reference graph, the family trio comprising a child and biological parents of the child, the method comprising: obtaining the sequence reads, the sequence reads having been previously obtained by sequencing biological samples obtained from each of the members of the family trio; obtaining at least one data structure storing data specifying nodes and edges of a family genomic reference graph, the family genomic reference graph having been previously generated; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, a set of variants for each of the members of the family trio.

Some aspects provide for at least one non-transitory computer-readable storage medium storing processor-executable instructions that, when executed by at least one computer hardware processor, causes the at least one computer hardware processor to perform a method for genotyping a family trio by using a family genomic reference graph and analyzing sequence reads from each member of the family trio using the family genomic reference graph, the family trio comprising a child and biological parents of the child, the method comprising: obtaining the sequence reads, the sequence reads having been previously obtained by sequencing biological samples obtained from each of the members of the family trio; obtaining at least one data structure storing data specifying nodes and edges of a family genomic reference graph, the family genomic reference graph having been previously generated; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, a set of variants for each of the members of the family trio.

Embodiments of any of the above aspects may have one or more of the following features.

Some embodiments further comprise: identifying, from among the updated plurality of variants, one or more de novo variants.

In some embodiments, identifying the one or more de novo variants comprises: identifying, based on results of aligning the at least some of the sequence reads to the family genomic reference graph, one or more variants that are detected in sequence reads obtained from a biological sample of the child and are not detected in sequence reads obtained from biological samples obtained from the biological parents of the child.

Some embodiments further comprise identifying a disease associated with the one or more de novo variants.

Some embodiments further comprise: identifying a plurality of variants based on the results of aligning the at least some of the sequence reads to the family genomic reference graph; and filtering the plurality of variants to obtain the updated plurality of variants, the filtering comprising for each particular variant of at least some of the plurality of variants: determining a coverage for the particular variant; and including the particular variant in the updated plurality of variants when the coverage is greater than a threshold coverage.

Some embodiments further comprise: identifying a plurality of variants based on the results of aligning the at least some of the sequence reads to the family genomic reference graph; and filtering the plurality of variants to obtain the updated plurality of variants, the filtering comprising for each particular variant of at least some of the plurality of variants: determining a confidence that a particular variant is present in a genome of the child and genomes of the biological parents of the child; and including the particular variant in the updated plurality of variants when the confidence exceeds a threshold confidence.

In some embodiments, the sequence reads include first sequence reads previously obtained by sequencing a first biological sample from a first biological parent of the child, second sequence reads previously obtained by sequencing a second biological sample from a second biological parent of the child, and third sequence reads previously obtained by sequencing a third biological sample from the child. In some embodiments, aligning the sequence reads to the initial genomic reference comprises aligning the first sequence reads, the second sequence reads, and the third sequence reads to the initial genomic reference. In some embodiments, identifying the initial plurality of variants comprises: identifying a first initial set of variants for the first biological parent based on results of aligning the first sequence reads to the initial genomic reference, identifying a second initial set of variants for the second biological parent based on results of aligning the second sequence reads to the initial genomic reference, and identifying a third initial set of variants for child based on results of aligning the third sequence reads to the initial genomic reference.

In some embodiments, aligning the at least some of the sequence reads to the family genomic reference graph comprises aligning, to the family genomic reference graph, at least some of the first sequence reads, at least some of the second sequence reads, and at least some of the third sequence reads.

In some embodiments, identifying the updated plurality of variants comprises: identifying, based on results of aligning the at least some of the first sequence reads to the family genomic reference graph, a first updated set of variants associated with the first biological parent; identifying, based on results of aligning the at least some of the second sequence reads to the family genomic reference graph, a second updated set of variants associated with the second biological parent; and identifying, based on results of aligning the at least some of the third sequence reads to the family genomic reference graph, a third updated set of variants associated with the child.

In some embodiments, identifying the updated plurality of variants comprises: identifying an intermediate plurality of variants based on the results of aligning the at least some of the sequence reads to the family genomic reference graph; identifying one or more Mendelian violations using the identified intermediate plurality of variants; and filtering the one or more Mendelian violations to identify the updated plurality of variants.

In some embodiments, the intermediate plurality of variants includes a first intermediate set of variants for the first biological parent, a second intermediate set of variants for the child, and a third intermediate set of variants for the second biological parent, and identifying the one or more Mendelian violations comprises: identifying first differences between haplotypes of the child and haplotypes of the first biological parent using the first intermediate set of variants and the third intermediate set of variants; identifying second differences between haplotypes of the child and haplotypes of the second biological parent using the second intermediate set of variants and the third intermediate set of variants; identifying one or more Mendelian violation loci based on the first differences and the second differences; and identifying the one or more Mendelian violations using the intermediate plurality of variants and the one or more Mendelian violation loci.

In some embodiments, identifying the updated plurality of variants comprises: joint genotyping the members of the family trio using the results of aligning the at least some of the sequence reads to the family genomic reference graph.

In some embodiments, generating the family genomic reference graph comprises: obtaining a linear genomic reference; and augmenting the linear genomic reference with variants in the initial set of variants for each of the members of the family trio.

In some embodiments, augmenting the linear genomic reference comprises representing the linear genomic reference as a graph having nodes and edges and augmenting the graph with one or more nodes and one or more edges representing at least some of the initial set of variants for each of the members of the family trio.

In some embodiments, the family genomic reference graph represents at least a portion of a human genome.

In some embodiments, the family genomic reference graph represents at least a chromosome of the human genome.

In some embodiments, the family genomic reference graph represents at least 10,000,000 nucleotides, at least 50,000,000 nucleotides, at least 100,000,000 nucleotides, at least 150,000,000 nucleotides, at least 200,000,000 nucleotides, or at least 250,000,000 nucleotides.

In some embodiments, the family genomic reference graph is a directed acyclic graph (DAG).

In some embodiments, the nodes and edges are encoded using elements in the at least one data structure, the nodes representing nucleotide sequences stored as respective strings of one or more symbols, and the edges including an edge representing a connection between at least two of the nodes.

In some embodiments, the at least one data structure comprises objects representing nodes and pointers representing edges, the objects comprising a first object representing a first node of the nodes, the first object storing at least one pointer representing at least one edge in the family genomic reference graph from the first node to one or more other nodes.

In some embodiments, the family genomic reference graph represents genomic information consisting of genomic information from the first parent, genomic information from the second parent, genomic information from the child, and genomic information represented by at least a portion of a linear genomic reference.

In some embodiments, aligning the sequence reads to the initial genomic reference comprises aligning the sequence reads to a population-specific genomic reference.

In some embodiments, the population-specific genomic reference comprises a population-specific genomic reference graph representing a linear reference sequence and population-specific variants relative to the linear reference sequence.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for genotyping a family trio including a child and biological parents of the child, according to some embodiments of the technology described herein.

FIG. 2 is a block diagram of an example system 200 for genotyping a family trio, according to some embodiments of the technology described herein.

FIG. 3A is a flowchart of an illustrative process 300 for genotyping a family trio, according to some embodiments of the technology described herein.

FIG. 3B is a flowchart of an illustrative process 320 for identifying an updated plurality of variants, according to some embodiments of the technology described herein.

FIG. 3C is a flowchart of another illustrative process 360 for identifying an updated plurality of variants, according to some embodiments of the technology described herein.

FIG. 4A is an illustrative example of genotyping a family trio, according to some embodiments of the technology described herein.

FIG. 4B is an illustrative example of identifying variants based on a result of aligning sequence reads to a family genomic reference graph, according to some embodiments of the technology described herein.

FIG. 4C is an illustrative example of identifying variants based on a result of aligning sequence reads to a family genomic reference graph and using joint genotyping, according to some embodiments of the technology described herein.

FIG. 4D is an illustrative example of aligning sequence reads to a genomic reference to identify variants, according to some embodiments of the technology described herein.

FIG. 4E is an illustrative example of generating a family genomic reference graph, according to some embodiments of the technology described herein.

FIG. 5A is a graph showing that genotyping a family trio over the entire genome, in accordance with embodiments of the technology described herein, is more accurate than genotyping a family trio in accordance with conventional techniques.

FIG. 5B is a graph showing that genotyping a family trio, in accordance with some embodiments of the technology described herein, results in a fewer number of spurious de novo variant calls as compared to conventional techniques.

FIG. 6A and FIG. 6B show that, as compared to conventional techniques, genotyping a family trio according to embodiments of the technology described herein reduces the number of spurious de novo variant calls, without increasing the number the number of missed de novo variant calls.

FIG. 7A is a graph showing that genotyping a family trio over the entire genome, in accordance with embodiments of the technology described herein, is more accurate than genotyping a family trio in accordance with conventional techniques.

FIG. 7B is a graph showing that genotyping a family trio, in accordance with embodiments of the technology described herein, results in a fewer number of spurious de novo variant calls as compared to conventional techniques.

FIGS. 8A and 8B show that genotyping a family trio, in accordance with embodiments of the technology described herein, results in fewer false negatives as compared to conventional techniques.

FIG. 9A and FIG. 9B show that genotyping a family trio, in accordance with embodiments of the technology described herein, results in a fewer number of missed and spurious rare variant calls as compared to conventional techniques.

FIG. 10 is a schematic diagram of an illustrative computing device with which aspects described herein may be implemented.

### DETAILED DESCRIPTION

The inventors have developed techniques for genotyping a family trio including a child and biological parents of the child. In some embodiments, the techniques for genotyping the family trio include (a) aligning sequence reads obtained from members of the family trio to an initial genomic reference (e.g., a linear or graph reference) to identify initial variants for each member of the family trio; (b) generating a family genomic reference graph using the identified initial variants; (c) aligning at least some of the sequence reads obtained from the members of the family trio to the family genomic reference graph; and (d) based on results of the aligning, identifying updated variants for members of the family trio. In some embodiments, the updated variants may be used to identify a disease for one or more of the members of the family trio.

Rare diseases are estimated to affect between 3.5-5.9% of the global population (about 263-446 million patients). As described above, the majority of rare diseases are caused by the presence of deleterious mutations in the patient's genome. The deleterious mutations are acquired from the genomes of the patient's biological parents following the rules of Mendelian inheritance (inherited variants), or acquired de novo, due to errors introduced during the process of reproduction (i.e., germline de novo variants).

Conventional germline de novo variant detection techniques involve (a) independently detecting variants in the genomes of a child and biological parents of the child, and (b) identifying, as germline de novo variants, variants that were solely detected in the genome of the child and not in the genomes of the parents. The inventors have recognized that these conventional techniques lack the sensitivity necessary for detecting germline de novo variants, and therefore cannot be used to accurately and efficiently detect and diagnose the rare and complex diseases that are caused by their presence. In particular, these conventional techniques are unequipped to handle sequencing errors and/or low-quality sequencing data obtained from one or more members of the family trio. When there are quality issues associated with the sequence reads obtained for one or both of the parents, inherited sequences may be detected for the child, but not for the parents even though they should be. Because the inherited sequences are solely detected for the child, the conventional techniques falsely identify them as germline de novo variants (i.e., spurious de novo variants). Because low sequencing quality is a frequent issue, and the sequencing is performed across at least 3 genomes (e.g., on the magnitude of over 9 billion base pairs), the conventional techniques output a large percentage (e.g., 90%) of spurious de novo variants relative to true de novo variants, making it challenging to identify the true de novo variants from among the reported variants. This, in turn, hinders the ability of the conventional techniques to accurately and efficiently identify a rare disease associated with the true de novo variants.

Accordingly, the inventors have developed techniques that address the above-described challenges associated with the conventional techniques for genotyping a family trio. In some embodiments, the techniques include (a) identifying initial variants for a family trio using an initial genomic reference (a linear reference or a graph reference, the graph reference may be a directed graph, for example, a directed acyclic graph or a directed graph with one or more cycles), (b) using the initial variants to generate a family-specific genomic reference (e.g., a graph reference embodied in a directed graph, for example, a directed acyclic graph or a directed graph with one or more cycles), and (c) using the family-specific genomic reference to identify an updated plurality of variants for the family trio. By accounting for variants that have already been identified for members of the family, the use of the family-specific genomic reference reduces bias that results from aligning sequence reads to a genomic reference that fails to represent family-specific variants and/or represents extra variants that are not prevalent in the family. Accordingly, use of the family-specific genomic reference enables a more accurate and sensitive identification of variants of a family trio, thereby reducing the number of spurious variants identified as compared to conventional techniques.

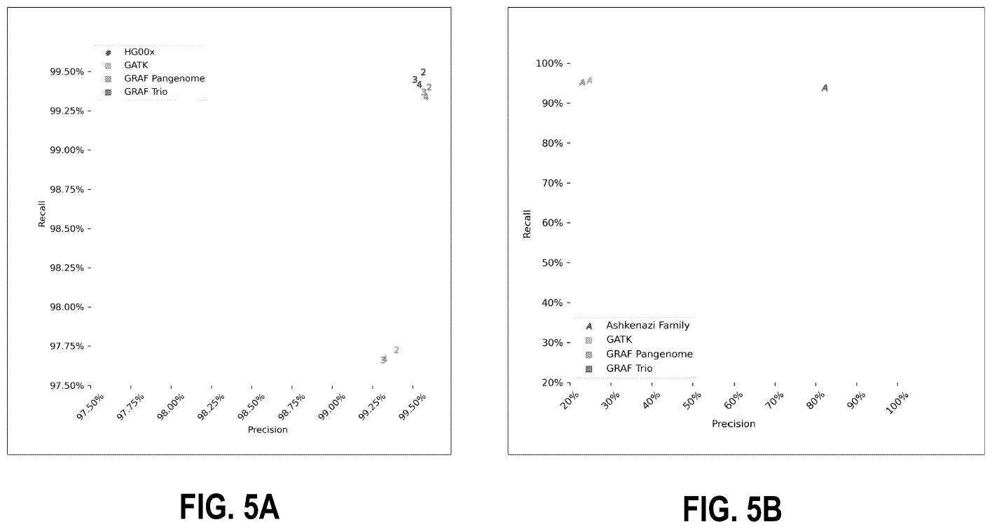

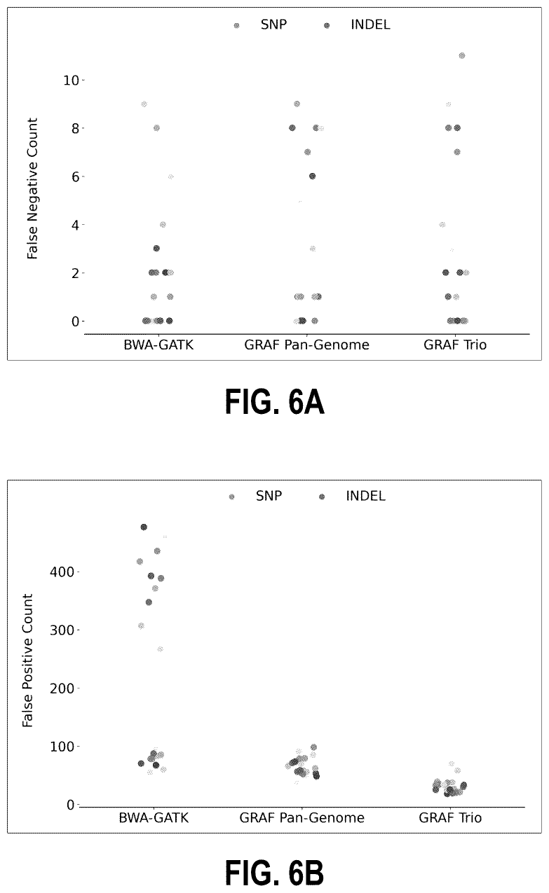

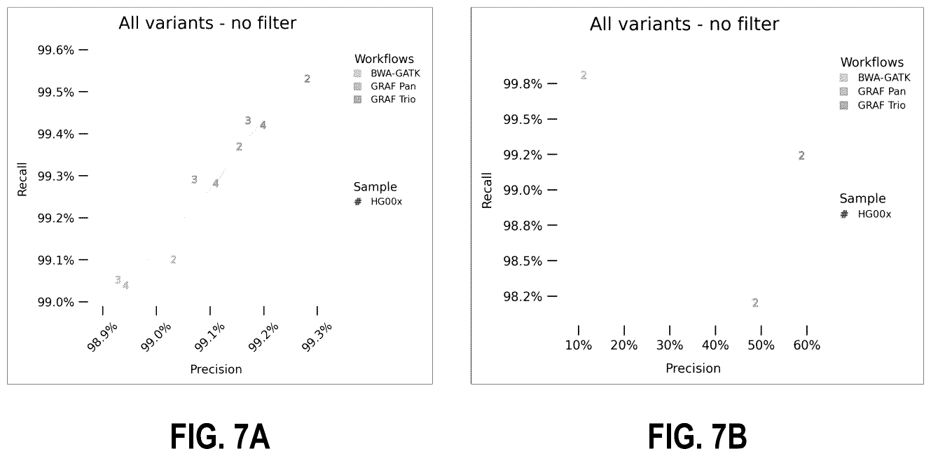

The improvement in accuracy is demonstrated in at least FIGS. 5A-7B, which show results of comparing the techniques developed by the inventors (“GRAF Trio”) to conventional techniques (“GRAF Pan-Genome” and “BWA-GATK”) for genotyping a family trio. The conventional techniques do not involve the use of a family-specific genomic reference for genotyping a family trio. FIG. 5A and FIG. 7A show that, as compared to the conventional techniques, the techniques developed by the inventors result in increased accuracy when genotyping a family trio over the entire genome. FIG. 5B and FIG. 7B show that the techniques developed by the inventors result in significant decrease in spurious de novo variant calls compared to the conventional techniques. Furthermore FIG. 6A and FIG. 6B, show that the techniques developed by the inventors not only decrease the number of spurious de novo variant calls (i.e., false positives), but they do so without increasing the number of missed de novo variant calls (i.e., false negatives). This is important because missing de novo variant calls would limit the ability of the techniques to accurately identify diseases associated with such missed variants.

In some embodiments, identifying the updated plurality of variants for the family trio includes identifying the presence of de novo variants in the child's genome. This includes, in some embodiments, identifying one or more variants that are inconsistent with Mendelian inheritance. In some embodiments, this includes (a) identifying differences between the child's haplotypes and the biological mother's haplotypes, (b) identifying differences between the child's haplotypes and the biological father's haplotypes, and (c) identifying the Mendelian violations based on the identified differences. In some embodiments, the techniques further include filtering the identified Mendelian violations based on a quality of the sequence and/or variant data. For example, Mendelian violations of low quality (e.g., below a threshold) may be excluded from further analysis. By identifying Mendelian violations (e.g., those that are not inherited from the parents) and filtering out low quality Mendelian violations, such techniques improve the accuracy of de novo variant identification by reducing false positives (e.g., spurious de novo variants) as compared to conventional techniques, as demonstrated in at least FIG. 5B.

In alternative embodiments, identifying the presence of de novo variants in the child's genome includes joint genotyping the family trio and using the results of the joint genotyping to identify the de novo variants. Joint genotyping refers to the process of (a) independently identifying potential variants for each member in the family trio based on the aligned positions of the individual's sequence reads relative to the family-specific genomic reference, and (b) using statistical techniques to refine the potential variants identified for each member of the family trio by considering the potential variants identified for the other members of the family trio. By sharing information across the members of the family trio, joint genotyping allows for the identification of variants in one or more of the members that might have otherwise been filtered out due to poor coverage of the variant and/or poor quality of the sequence reads. Accordingly, the techniques developed by the inventors are equipped to handle low-quality sequencing data obtained from one or more members of the family trio, and therefore return a reduced number of spurious de novo mutations relative to the conventional techniques, as demonstrated in at least FIG. 7B.

Following below are descriptions of various concepts related to, and embodiments of, techniques for genotyping a family trio. It should be appreciated that various aspects described herein may be implemented in any of numerous ways, as the techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

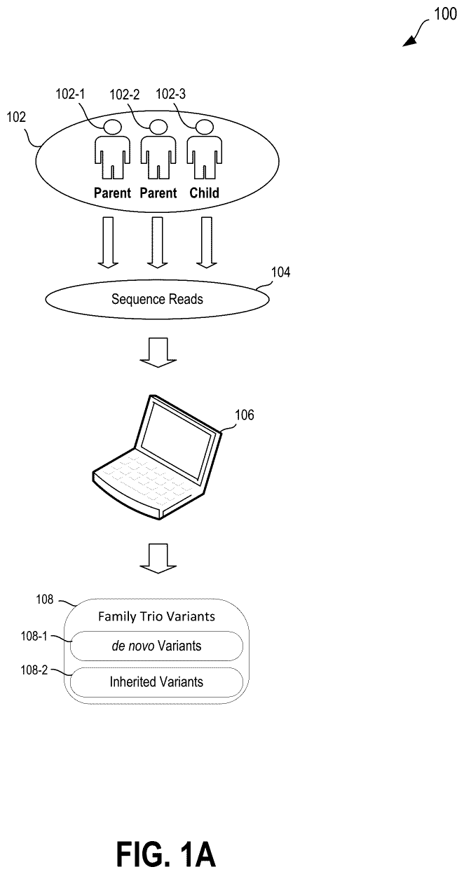

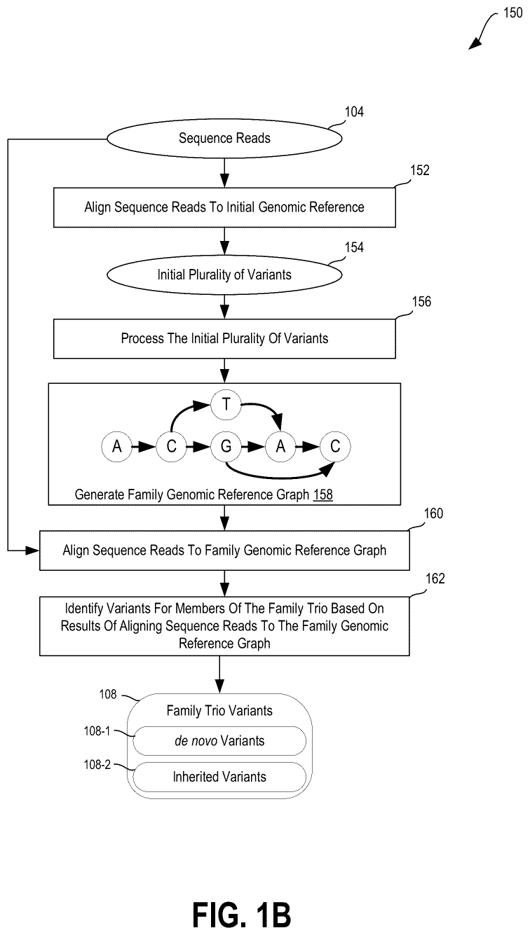

FIG. 1A is a diagram depicting an illustrative technique 100 for genotyping a family trio including a child and biological parents of the child, according to some embodiments of the technology described herein. Technique 100 includes obtaining sequence reads 104 from family trio 102 and processing the sequence reads 104 using computing device 106 to obtain family trio variants 108. In some embodiments, at least some of the family trio variants 108 (e.g., the de novo variants) are used to identify a disease associated with one or more members of the family trio 102.

In some embodiments, aspects of the illustrated technique 100 may be implemented in a clinical or laboratory setting. For example, aspects of the illustrated technique 100 may be implemented on a computing device 106 that is located within a clinical or laboratory setting. In some embodiments, the computing device 106 may obtain sequence reads 104 from a sequencing platform co-located with the computing device 106 within the clinical or laboratory setting. For example, the computing device 106 may be included within the sequencing platform. In some embodiments, the computing device 106 may indirectly obtain the sequence reads 104 from a sequencing platform that is located externally from or co-located with the computing device 106 within the clinical or laboratory setting. For example, the computing device 106 may obtain the sequence reads 104 via at least one communication network, such as the Internet or any other suitable communication network(s), as aspects of the technology are not limited in this respect.

In some embodiments, aspects of the illustrated technique 100 may be implemented in a setting that is located externally from a clinical or laboratory setting. In this case, the computing device 106 may indirectly obtain sequence reads 104 from a sequencing platform located within or externally to a clinical or laboratory setting. For example, the sequence reads 104 may be provided to the computing device 106 via at least one communication network, such as the Internet or any other suitable communication network(s), as aspects of the technology described herein are not limited in this respect.

As shown in FIG. 1A, sequence reads 104 are obtained from family trio 102. For example, the sequence reads 104 may include sequence reads from each member of the family trio 102. The family trio may include a child 102-3, biological parent 102-1, and biological parent 102-2. Additionally, sequence reads 104 may be obtained from one or more other biological relatives of child 102-3. For example, in addition to sequence reads from a child's biological parents, the sequence reads 104 may be obtained from any sibling(s) of child 102-3, one or more of the maternal grandparents of child 102-3, one or more of the paternal grandparents of child 102-3, and/or any other direct line ancestors of child 102-3.

In some embodiments, the sequence reads 104 are obtained by processing biological sample(s) obtained from the member(s) of the family trio 102. In some embodiments, the biological sample includes a germline sample such as, for example, a blood sample and/or a saliva sample. Germline samples may refer to samples that include cells which have only had a short time to accumulate somatic mutations (e.g., acquired during ageing and cell division), since they are constantly renewed. In some embodiments, when the germline sample is a blood sample, the blood sample includes buffy coat. Buffy coat refers to the layer of intermediate cell density resulting from centrifugal separation of blood tissue. This layer is enriched in plasma lymphocyte cells, which are constantly renewed. In some embodiments, the origin, type, or preparation methods of the biological sample(s) may include any of the embodiments described the section “Biological Samples.”

In some embodiments, the sequence reads 104 are obtained using a sequencing platform such as a next generation sequencing platform (e.g., Illumina®, Roche®, Ion Torrent®, etc.), or any high-throughput or massively parallel sequencing platform. In some embodiments, these methods may be automated, in some embodiments, there may be manual intervention. In some embodiments, the sequence reads 104 may be the result of non-next generation sequencing (e.g., Sanger sequencing).

The sequence reads 104 may include DNA sequence reads, DNA exome sequence reads (e.g., reads obtained from whole exome sequencing (WES)), DNA genome sequence reads (e.g., reads obtained from whole genome sequencing (WGS)), gene sequence reads, bias-corrected sequence reads, or any other suitable type of sequence reads obtained from a sequencing platform and/or derived from data obtained from a sequencing platform. In some embodiments, the origin, type, or preparation methods of the sequence reads may include any of the embodiments described the section “Sequencing Data.”

In some embodiments, a computing device 106 is used to process the sequence reads 104 to obtain the family trio variants 108. The computing device 106 may be operated by a user such as a doctor, clinician, researcher, a member of the family trio 102, and/or any other suitable entity. For example, the user may provide the sequence reads 104 as input to the computing device 106 (e.g., by uploading a file), provide user input specifying processing or other methods to be performed using the sequence reads 104, and/or provide input specifying one or more clinical features associated with one or more members of family trio 102.

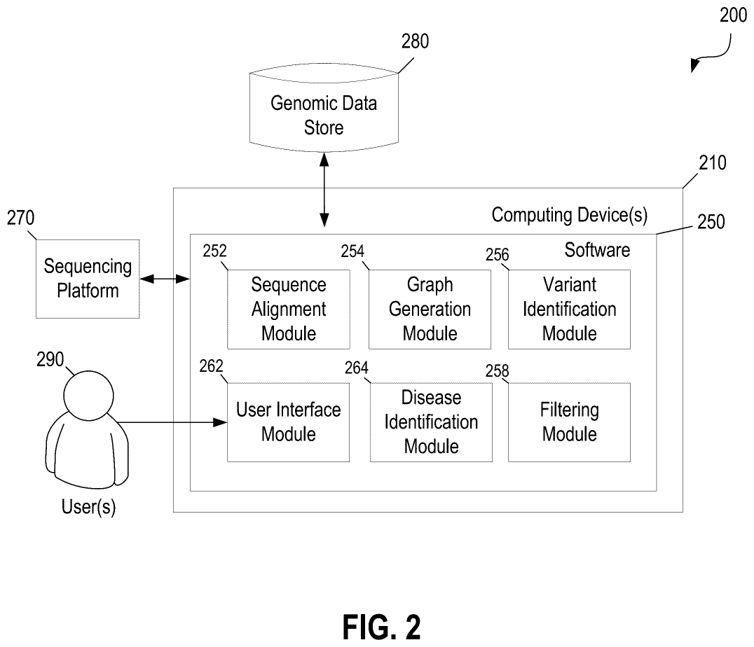

In some embodiments, software on computing device 106 may be used to identify family trio variants 108 for one or more members of the family trio 102 and/or identify a disease (e.g., a rare disease) for one or more members of the family trio 102. An example of computing device 106 and such software is described herein including at least with respect to FIG. 2 (e.g., computing device(s) 210 and software 250). In some embodiments, software on the computing device 106 may be configured to process at least some (e.g., all) of the sequence reads 104 to identify the family trio variants 108. In some embodiments, this may include: (a) aligning the sequence reads 104 to an initial genomic reference to obtain an initial plurality of variants, (b) generating a family genomic reference graph, (d) aligning at least some of the sequence reads 104 to the family genomic reference graph to obtain the family trio variants 108 (e.g., an updated plurality of variants). Example techniques for identifying variants for one or more members of a family trio are described herein including at least with respect to FIGS. 1B, 3A-3C, and 4A-4E.

In some embodiments, software on the computing device 106 may additionally, or alternatively, identify rare and/or de novo variants from among the family trio variants 108. For example, the family trio variants 108 may include inherited variants 108-2 and/or de novo variants 108-1, at least some of which may include rare variants. The software may identify de novo variants by identifying variants that were only identified for the child of the family trio 102, and not for either of the parents. The software may identify rare variants by identifying variants having an allele frequency less than or equal to a threshold allele frequency. Additionally, or alternatively, in some embodiments, software on the computing device 106 may use the variants 108 identified for the member(s) of the family trio 102 to identify a disease associated with the variants 108.

In some embodiments, the computing device 106 is configured to generate an output indicating one or more variants and/or diseases identified for member(s) of the family trio 102. For example, the output may indicate one or more germline de novo variants that occurred in child 102-3 of family trio 102 during the process of reproduction. Additionally, or alternatively, output may indicate one or more other variants such as those shared by one or more members of the family trio 102 (e.g., a variant of one or both of parent 102-1 and parent 102-2, which was inherited by child 102-3). Additionally, or alternatively, the output may indicate one or more diseases associated with one or more variants identified for the family trio 102. For example, the output may indicate a rare disease associated with one or more of the family trio variants 108.

In some embodiments, the output of computing device 106 (e.g., the family trio variants 108) is stored (e.g., in memory), displayed via a user interface, transmitted to one or more other devices, used to generate a report, or otherwise processed using any other suitable techniques, as aspects of the technology are not limited in this respect. For example, the output of computing device 106 may be displayed using a graphical user interface (GUI) of a computing device (e.g., computing device 106).

In some embodiments, the output of the computing device 106 may be in the form of a report, such as a report including an indication of one or more variants (e.g., the family trio variants 108, etc.) and/or an indication of one or more diseases associated with variant(s) identified for member(s) of the family trio 102. The generated report can provide a summary of information, so that a clinician can identify genetic variant(s) and/or disease(s) associated with one or more members of the family trio 102. The report as described herein may be a paper report, an electronic record, or a report in any format that is deemed suitable in the art. The report may be shown and/or stored on a computing device known in the art (e.g., a handheld device, desktop computer, smart device, website, etc.). The report may be shown and/or stored on any device that is suitable as understood by a skilled person in the art.

In some embodiments, methods disclosed herein can be used for commercial diagnostic purposes. For example, the generated report may include, but is not limited to, information concerning sequencing data (e.g., sequence reads 104), clinical and pathological factors, subject's prognostic analysis, and/or other information. In some embodiments, the methods and reports may include database management for the keeping of the generated reports. For instance, the methods as disclosed herein can create a record in a database for one or more members of the family trio 102 and populate the specific record with data for the subject. In some embodiments, the generated report can be provided to the member(s) of the family trio 102 and/or to the clinicians. In some embodiments, a network connection can be established to a server computer that includes the data and report for receiving or outputting. In some embodiments, the receiving and outputting of the data or report can be requested from the server computer.

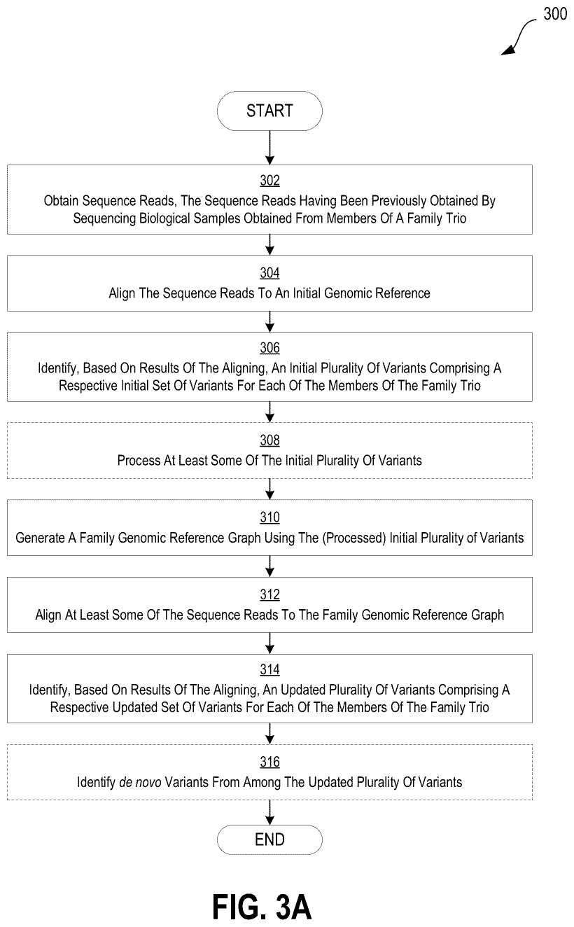

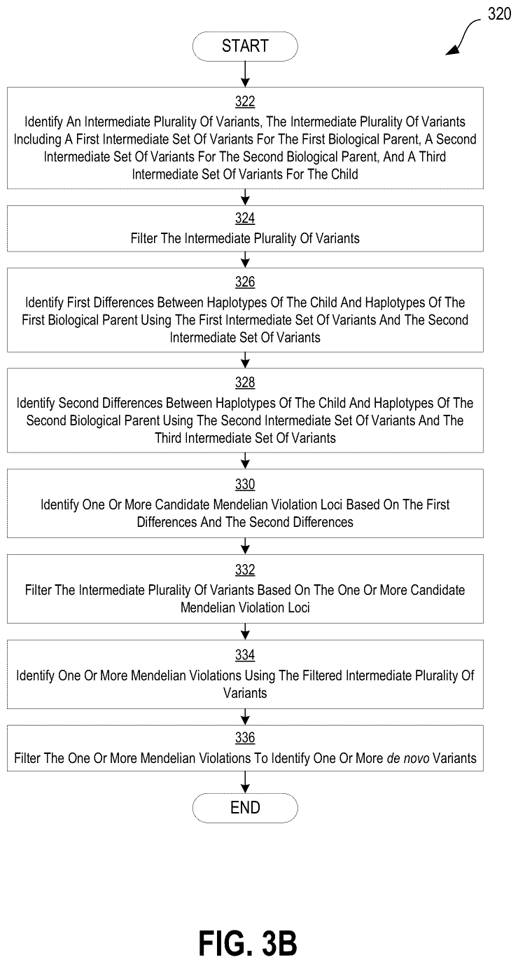

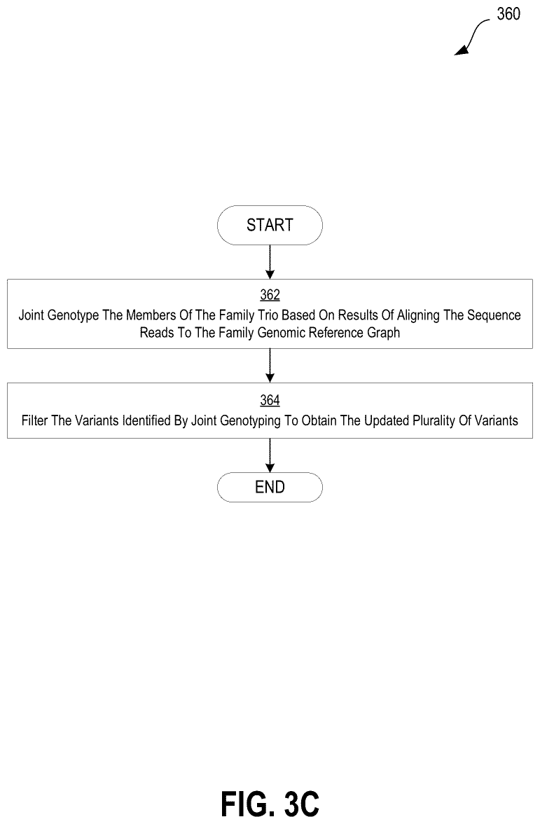

In some embodiments, the computing device 106 includes one or multiple computing devices. In some embodiments, when the computing device 106 includes multiple computing devices, each of the computing devices may be used to perform the same process or processes. For example, each of the multiple computing devices may include software used to implement process 300 shown in FIG. 3A, process 320 shown in FIG. 3B, and/or process 360 shown in FIG. 3C. In some embodiments, when the computing device 106 includes multiple computing devices, the computing devices may be used to perform different processes or different aspects of a process. For example, one computing device may include software used to align sequence reads to a reference data structure (e.g., an initial reference sequence, a reference graph, etc.), while a different computing device may include software used to identify variants based on aligning the sequence reads to the reference data structure.

In some embodiments, when the computing device 106 includes multiple computing devices, the multiple computing devices may be configured to communicate via at least one communication network such as the Internet or any other suitable communication network(s), as aspects of the technology described herein are not limited in this respect. For example, one computing device may be configured to align sequence reads to a reference data structure, and then provide results of the alignment to one or more other computing devices via the communication network.

FIG. 1B is a diagram depicting an illustrative technique 150 for processing sequence reads 104 to identify the family trio variants 108. The illustrative technique 150 includes (a) at act 152, aligning sequence reads to an initial genomic reference to obtain the initial plurality of variants 154; (b) at act 156, processing the initial plurality of variants 154; (c) at act 158, using the initial plurality of variants 154 to generate the family genomic reference graph; (c) at act 160, aligning at least some of the sequence reads 104 to the family genomic reference graph; and (d) at act 162, identifying variants 108 for the members of the family trio based on resulting of aligning the sequence reads to the family genomic reference graph. As described herein, including at least with respect to FIG. 1A, technique 150 may be implemented using a computing device such as computing device 106 shown in FIG. 1A.

As shown in FIG. 1B, illustrative technique 150 includes aligning sequence reads 104 to an initial genomic reference at act 152. The initial genomic reference may include any genomic reference suitable for genotyping a subject such as one or more members of family trio 102, as aspects of the technology described herein are not limited in this respect. For example, in some embodiments, the initial genomic reference includes a linear genomic reference. The linear genomic reference may include a human genome reference sequence such as, for example, human genome version 19 (hg19), hg38, Genome Reference Consortium human reference 38 (GRCh38), GRCh37, or any other suitable human genome reference sequence.

In some embodiments, the initial genomic reference includes a genomic reference graph representing a linear reference sequence having nodes and edges and edges. The genomic reference graph may be one or more data structures that specify nodes and edges connecting the nodes. The nodes may represent nucleotide sequences stored as respective strings of one or more symbols, and the edges may represent a connection between at least two of the nodes. Alternatively, the edges may represent nucleotide sequences stored as respective strings of one or more symbols, and the nodes may represent a connection between at least two of the edges. In some embodiments, the data structure includes objects that represent the nodes and pointers that represent the edges. As one non-limiting example, the data structure may be a directed acyclic graph (DAG). Example techniques for generating a genomic reference graph are described by Rakocevic, G., et al. (“Fast and accurate genomic analysis using genome graphs.” Nat Genet. 51.2 (2019): 354-362), which is incorporated by reference herein in its entirety.

Additionally, or alternatively, in some embodiments, the initial genomic reference includes a genomic reference graph representing a linear reference sequence and variation of the linear reference sequence. For example, such a genomic reference graph may be generated by representing a linear genomic reference as a graph having nodes and edges and augmenting the linear genomic reference with one or more nodes and one or more edges representing at least some variants.

In some embodiments, the initial genomic reference graph is specific to one or more populations. Such a reference graph may represent variants that are common among members of the one or more populations. For example, the initial genomic reference graph may represent variants that are common among members of the one or more populations to which the members of the family trio (e.g., family trio 102) belong. Nonlimiting examples of populations include African ancestry (AFR), American ancestry (AMR), South-Asian ancestry (SAS), Eastern-Asian ancestry (EAS), and European ancestry (EUR). Variants that are specific to particular populations may be obtained from any suitable source such as, for example, the 1000 Genomes Project consortium. The population(s) to which the members of the family trio belong may be identified using any suitable techniques, as aspects of the technology are not limited in this respect. Example techniques for generating a population-specific genomic reference graph are described by Tetikol, H. S., et al. (“Pan-African genome demonstrates how population-specific genome graphs improve high-throughput sequencing data analysis.” Nature Communications 13.1 (2022): 4384), which is incorporated by reference herein in its entirety.

In some embodiments, when the initial genomic reference is a linear genomic reference (e.g., represented as a graph or not), sequence reads 104 may be aligned to the linear genomic reference, at act 152, using any suitable linear alignment techniques, as aspects of the technology described herein are not limited in this respect. In some embodiments, the alignment may be performed using dynamic programming. Nonlimiting examples of linear alignment techniques include, but are not limited to, the Needleman-Wunsch algorithm, the Smith-Waterman algorithm, and Burrows-Wheeler Alignment (BWA), among others. The Needleman-Wunsch algorithm is described by Needleman, S. and Wunsch, C. (“A general method applicable to the search for similarities in the amino sequence of two proteins.” Journal of molecular biology 48.3 (1970): 443-453), which is incorporated by reference herein in its entirety. The Smith-Waterman algorithm is described by Smith, T. F. and Waterman, M. S. (“Identification of Common Molecular Subsequences.” Journal of molecular biology 147.1 (1981): 195-197), which is incorporated by reference herein in its entirety. BWA is described by Li, H. and Durbin, R. (“Fast and accurate short read alignment with Burrows-Wheeler transform.” Bioinformatics. 25.14 (2009): 1754-1760), which is incorporated by reference herein in its entirety.

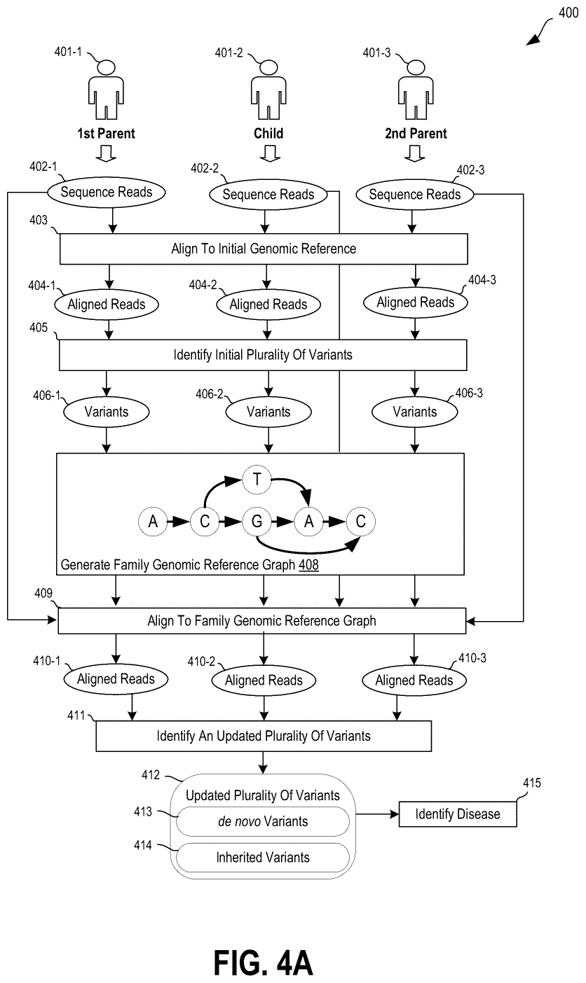

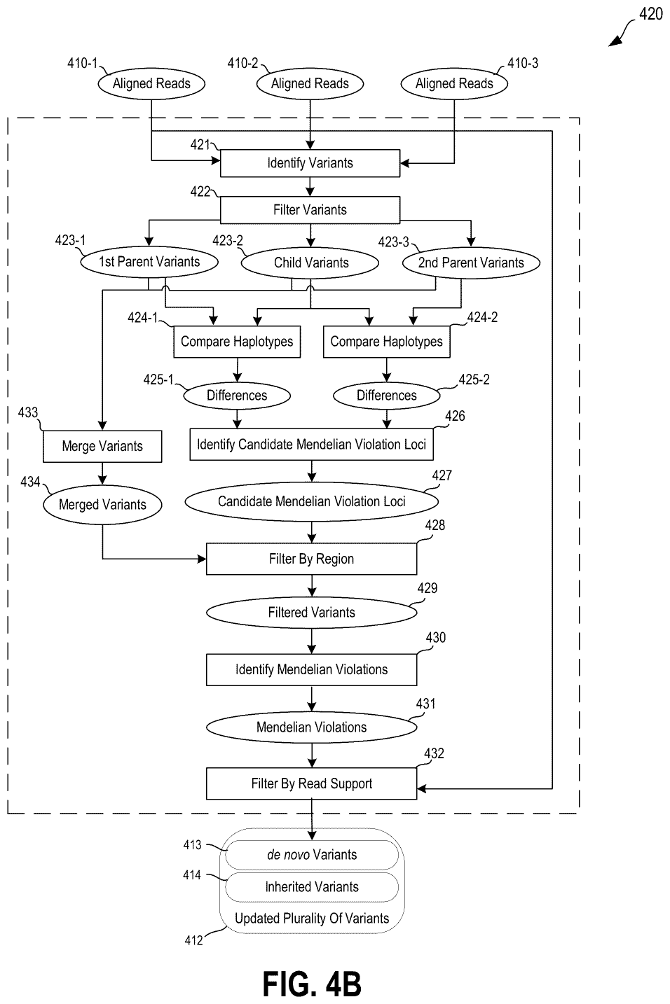

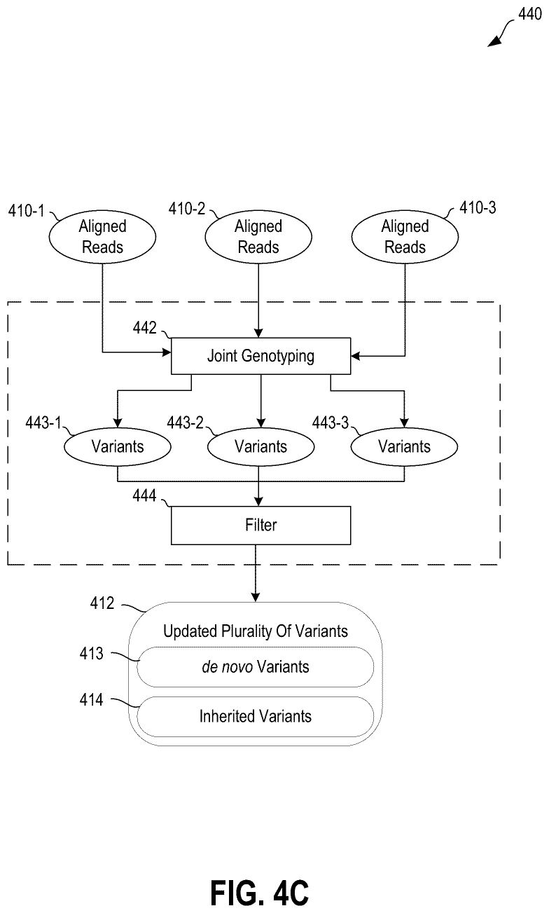

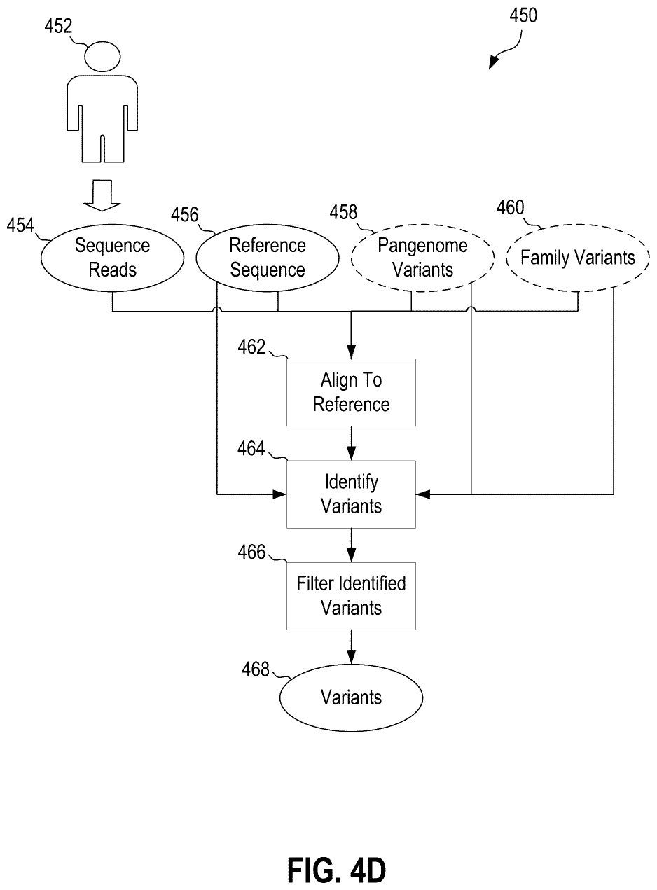

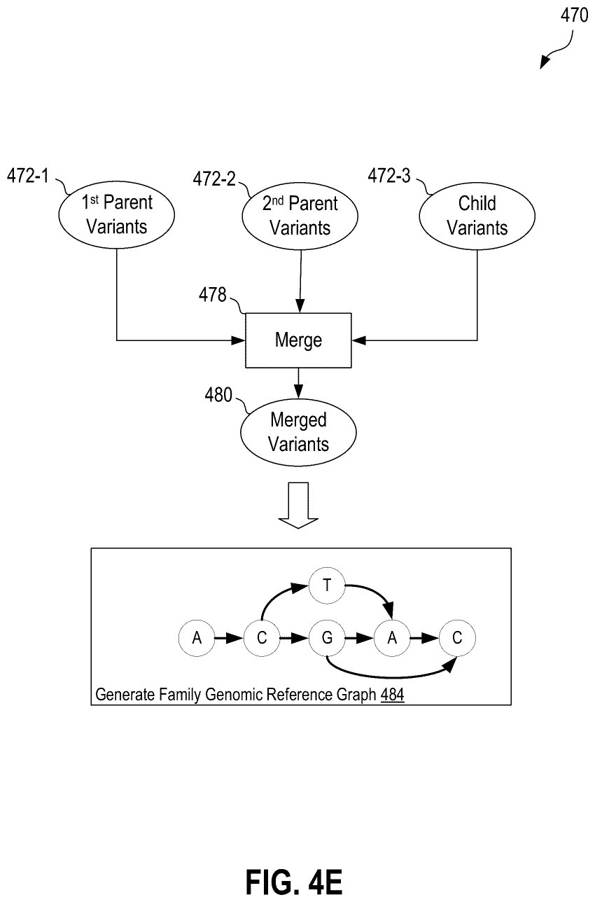

In some embodiments, when the initial genomic reference is a genomic reference graph representing a linear genomic reference and variation thereof, sequence reads 104 may be aligned to the genomic reference graph, at act 152, using any suitable graph alignment techniques, as aspects of the technology described herein are not limited in this respect. In some embodiments, the graph alignment may be performed using dynamic programming. In some embodiments, the graph alignment technique may include a linear alignment technique that has been modified to handle the branches and merges present in a genomic reference graph. Example graph alignment techniques are described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”, each of which is incorporated by reference herein in its entirety. Examples of aligning sequence reads to a genomic reference graph are further described herein including at least with respect to FIG. 4B.

In some embodiments, an initial plurality of variants 154 is identified as a result of aligning the sequence reads 104 to the initial genomic reference at act 152. The initial plurality of variants 154 includes an initial set of variants for each member of the family trio (e.g., family trio 102 shown in FIG. 1A). For example, the initial plurality of variants may include a set of variants for a child (e.g., child 102-3) and a set of variants for each of the biological parents (e.g., parent 102-1 and parent 102-2) of the child. The initial plurality of variants 154 may be in any suitable format, as embodiments of the technology described herein are not limited in this respect. For example, the initial plurality of variants 154 may be in variant call format (VCF).

In some embodiments, identifying a set of variants for an individual includes identifying where the aligned sequence reads for that individual differs from the genomic reference. In some embodiments, this is performed using variant calling software. Nonlimiting examples of variant calling software include GRAF Variant Caller software, Genomic Atlas Toolkit (GATK) software, SAMtools software, BCFtools software, or any other suitable variant calling software as aspects of the technology described herein are not limited in this respect. GATK software is described by Van der Auwera G A & O'Connor B D. (“Genomics in the Cloud: Using Docker, GATK, and WDL in Terra (1st Edition)”. O'Reilly Media. (2020)), which is incorporated by reference herein in its entirety. SAMtools software is described by Li, H., et al. (“The sequence alignment/map format and SAMtools.” Bioinformatics 25.16 (2009): 2078-2079), which is incorporated by reference herein in its entirety. BCFtools is described by Li H. (“A statistical framework for SNP calling, mutation discovery, association mapping and population genetical parameter estimation from sequencing data.” Bioinformatics (2011) 27 (21) 2987-93), which is incorporated by reference herein in its entirety.

In some embodiments, the initial plurality of variants 154 is processed, at act 156, prior to being used to generate the family genomic reference graph at act 158. In some embodiments, processing the initial plurality of variants 154 includes processing each set of variants for each member of the family trio. For example, this may include processing the set of variants for the child and processing the set of variants for each biological parent of the child.

In some embodiments, processing a set of variants includes normalizing the set of variants. Normalizing the set of variants may include left-aligning the set of variants (e.g., left-aligning insertion-deletions (indels)), which refers to shifting the start positions of the variants to the left. Additionally, or alternatively, normalizing a set of variants may include representing each variant in as few nucleotides as possible without reducing the length of any allele to zero, such that the variants are parsimonious. Additionally, or alternatively, normalizing a set of variants may include determining whether the reference alleles match the reference sequence. Additionally, or alternatively, normalizing a set of variants may include splitting multiallelic sites into multiple rows and/or recovering multiallelics from multiple rows. In some embodiments, normalizing the sets of variants may include using one or more software tools such as, for example, the “BCFtools norm” software tool. BCFtools is described by Li H. (“A statistical framework for SNP calling, mutation discovery, association mapping and population genetical parameter estimation from sequencing data.” Bioinformatics (2011) 27 (21) 2987-93), which is incorporated by reference herein in its entirety.

In some embodiments, processing a set of variants additionally or alternatively includes filtering the set of variants. In some embodiments, filtering the set of variants may include applying one or more fixed threshold filters to the one or more variants included in the set of variants. Additionally, or alternatively, filtering the set of variants may include identifying clusters of indels separated by fewer than or equal to a threshold number of base pairs, and excluding all but one of the indels from subsequent processing. Additionally, or alternatively, any other suitable filtering techniques may be used to filter a set of variants, as embodiments of the technology described herein are not limited in this respect. In some embodiments, filtering the set of variants may include using one or more software tools such as, for example, the “BCFtools filter” software tool.

In some embodiments, processing the initial plurality of variants 154 additionally, or alternatively, includes merging the sets of variants obtained for each member of the family trio. For example, this may include merging the set of variants obtained for a child with the sets of variants obtained for each of the biological parents of the child to generate a merged set of variants. In some embodiments, merging the sets of variants includes merging multiple VCF files to generate a single, merged VCF file. The sets of variants may be merged using one or more software tools such as, for example, the “BCFtools merge” software tool.

In some embodiments, the initial plurality of variants 154 is used to generate the family genomic reference graph at act 158. In some embodiments, the family genomic reference graph is generated at least in part by augmenting a linear reference with the initial plurality of variants 154 (e.g., the processed initial plurality of variants 154). For example, the linear reference may be represented by nodes connected by edges. The nodes may represent nucleotide sequences stored as respective strings of one or more symbols, and the edges may represent a connection between at least two of the nodes. Alternatively, the edges may represent nucleotide sequences stored as respective strings of one or more symbols, and the nodes may represent a connection between at least two of the edges. The linear reference may be augmented by including, at one or more positions along the linear reference, alternative nodes and/or edges, thereby generating alternative paths through a genomic graph reference. For example, node(s) may be used to represent an insertion at the position and an edge may be used to represent a deletion. Example techniques for generating a genomic reference graph are described by Rakocevic, G., et al. (“Fast and accurate genomic analysis using genome graphs.” Nat Genet. 51.2 (2019): 354-362).

The family genomic reference graph may represent any suitable number of nucleotides, as aspects of the technology described herein are not limited in this respect. For example, the family genomic reference graph may represent a number of nucleotides between 10 and 3 billion nucleotides, between 1,000 and 2 billion nucleotides, between 10,000 and 1 billion nucleotides, between 100,000 and 100 million nucleotides, between 1 million and 10 million nucleotides, or any other suitable number of nucleotides. Additionally, or alternatively, the family genomic reference graph may represent at least 10, at least 100, at least 1,000, at least 10,000, at least 100,000, at least 1 million, at least 10 million, at least 50 million, at least 100 million, at least 150 million, at least 200 million, at least 250 million, or at least any other suitable number of nucleotides. Additionally, or alternatively, the family genomic reference graph may represent at most 3 billion, at most 2 billion, at most 1 billion, at most 250 million, at most 150 million, at most 100 million, at most 50 million, at most 10 million, at most 1 million, or at most any other suitable number of nucleotides. It should be appreciated that any of the above-listed upper bounds may be coupled with any of the above-lister lower bounds.

In some embodiments, at least some (e.g., all) of the sequence reads 104 are aligned to the family genomic reference graph, at act 160. For example, in some embodiments, at least some of the sequence reads obtained for the child (e.g., child 102-3 in FIG. 1A) are aligned to the family genomic reference graph. Additionally, or alternatively, at least some of the sequence reads obtained for a first of the two biological parents of the child (e.g., parent 102-1 in FIG. 1A) are aligned to the family genomic reference graph. Additionally, or alternatively, at least some of the sequence reads obtained for a second of the two biological parents of the child (e.g., parent 102-2 in FIG. 1A) are aligned to the family genomic reference graph. Techniques for aligning sequence reads to a genomic reference graph are described herein including at least with respect to act 152 of illustrative technique 150.

At act 162, variants are identified for members of the family trio based on results of aligning the sequence reads to the family genomic reference graph. In some embodiments, identifying the variants includes (a) identifying variants for each member of the family trio using results of aligning the sequence reads to the family genomic reference graph, (c) comparing the child's haplotypes with those of the biological parents using the identified variants, (d) identifying candidate Mendelian violation loci based on results of the comparing, and (e) identifying the family trio variants (e.g., de novo variants) using the variants identified at act (a) and the candidate Mendelian violation loci. In some embodiments, identifying variants at act 162 additionally, or alternatively, includes one or more steps for filtering the variants. Example techniques for identifying the family variants are described herein including at least with respect to act 314 of process 300 shown in FIG. 3A, process 320 shown in FIG. 3B, and example 420 shown in FIG. 4B. In alternative embodiments, identifying the variants at act 162 includes joint genotyping (or joint variant calling) the members of the family trio (e.g., family trio 102) based on results of aligning the sequence reads to the family genomic reference graph, and filtering the variants identified by joint genotyping. Example techniques for identifying variants using joint genotyping and filtering are described herein including at least with respect to process 360 shown in FIG. 3C and example 440 shown in FIG. 4C.

In some embodiments, the de novo variants 168 are identified from among the family trio variants 108. For example, the de novo variants 168 may be identified as variants that are included in the set of variants identified for the child but are not included in the sets of variants identified for either of the biological parents.

FIG. 2 is a block diagram of an example system 200 for genotyping a family trio, according to some embodiments of the technology described herein. System 200 includes computing device(s) 210 configured to have software 250 execute thereon to perform various functions in connection with genotyping a family trio and/or identifying a disease for member(s) of the family trio. In some embodiments, software 250 includes a plurality of modules. A module may include processor-executable instructions that, when executed by at least one computer hardware processor, cause the at least one computer hardware processor to perform the function(s) of the module. Such modules are sometimes referred to herein as “software modules,” each of which includes processor executable instructions configured to perform one or more processes, such as process 300 described herein including at least with respect to FIG. 3A, process 320 described herein including at least with respect to FIG. 3B, and process 360 described herein including at least with respect to FIG. 3C.

The computing device(s) 210 may be operated by one or more user(s) 290. For example, the user(s) 290 may include one or more individuals who are treating and/or studying (e.g., doctors, clinicians, researchers, etc.) one or more members of the family trio. Additionally, or alternatively, the user(s) 290 may include one or more members of the family trio being genotyped. In some embodiments, the user(s) 290 may provide, as input to the computing device(s) 210 (e.g., by uploading one or more files, by interacting with a user interface of the computing device(s) 210, etc.) sequence reads obtained for one or more members of the family trio (e.g., previously-obtained from the members of the family trio). Additionally, or alternatively, the user(s) 290 may provide input specifying processing or other methods to be performed on the sequence reads. Additionally, or alternatively, the user(s) 290 may access results of processing the sequence reads. For example, the user(s) 290 may access results of genotyping one or more members of the family trio (e.g., information specifying de novo variants, inherited variants, etc.).

As shown in FIG. 2, software 250 includes multiple software modules for genotyping members of a family trio and/or identifying a disease for members of a family trio. Such software modules include a sequence alignment module 252, a graph generation module 254, a variant identification module 256, a filtering module 258, a disease identification module 264.

In some embodiments, the sequence alignment module 252 obtains sequence reads (e.g., sequence reads 104 shown in FIGS. 1A-1B) from sequencing platform 270, the user(s) 290 (e.g., by the user(s) uploading the sequence reads), and/or the genomic data store 280. In some embodiments, the sequence alignment module 252 obtains one or more genomic references from user(s) 290 (e.g., by the user(s) uploading the genomic references), from the graph generation module 254, and/or from genomic data store 280.

In some embodiments, the sequence alignment module 252 is configured to align the sequence reads to a genomic reference. For example, in some embodiments, the sequence alignment module 252 may be configured to align the sequence reads to an initial genomic reference. As described herein, including with respect to FIG. 1B, the initial genomic reference may include a linear genomic reference or a genomic reference graph. Additionally, or alternatively, in some embodiments, the sequence alignment module 252 may be configured to align the sequence reads to a family genomic reference graph. For example, as described herein, the family genomic reference graph may represent a linear reference and genetic variants of the linear reference that have been identified as present in the genome(s) of one or more members of the family trio. In some embodiments, the sequence alignment module 252 may be configured to receive a family genomic reference graph from the graph generation module 254.

In some embodiments, the sequence alignment module 252 is configured to perform an alignment algorithm to align the sequence reads to the genomic reference. As described herein, the alignment algorithm may depend on the type of genomic reference (e.g., linear or graph) to which the sequence reads are being aligned. When the genomic reference is a linear genomic reference, then the sequence alignment module 252 may be configured to perform any alignment algorithm suitable for aligning sequence reads to a linear genomic reference, as aspects of the technology described herein are not limited in this respect. Nonlimiting examples of linear alignment algorithms include, but are not limited to, the Needleman-Wunsch algorithm, the Smith-Waterman algorithm, and Burrows-Wheeler Alignment (BWA), among others. When the genomic reference is a genomic reference graph representing a linear genomic reference and variation thereof, then the sequence alignment module 252 may be configured to perform any alignment algorithm suitable for aligning sequence reads to a genomic reference graph, as aspects of the technology described herein are not limited in this respect. Nonlimiting examples of graph alignment algorithms include, but are not limited to, the alignment algorithms described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”.

In some embodiments, the variant identification module 256 obtains sequence alignment results from the sequence alignment module 252, genomic data store 280, and/or user(s) 290 (e.g., by uploading the sequence alignment results). The sequence alignment results may identify one or more positions of a genomic reference to which sequence reads (e.g., sequence reads from member(s) of the family trio) align.

In some embodiments, the variant identification module 256 is configured to identify an initial plurality of variants for the members of the family trio based on the results of aligning the sequence reads obtained for the family trio to an initial genomic reference. In some embodiments, identifying a set of variants for an individual includes identifying where the aligned sequence reads for that individual differs from the genomic reference. In some embodiments, the variant identification module 256 uses variant calling software to identify variants based on the alignment results. Nonlimiting examples of variant calling software include GRAF Variant Caller software, Genomic Atlas Toolkit (GATK) software, SAMtools software, BCFtools software, or any other suitable variant calling software as aspects of the technology described herein are not limited in this respect.

In some embodiments, the variant identification module 256 is configured to identify an updated plurality of variants for the members of the family trio based on results of aligning the sequence reads obtained for the family trio to a family genomic reference graph. In some embodiments, this includes identifying de novo variants for the child and/or variants that were inherited by the child from at least one of the biological parents.

In some embodiments, to identify the updated plurality of variants, the variant identification module 256 may use variant calling software to identify variants based on sequence reads aligned to the family genomic reference graph. Nonlimiting examples of variant calling software include GRAF Variant Caller software, Genomic Atlas Toolkit (GATK) software, SAMtools software, BCFtools software, or any other suitable variant calling software as aspects of the technology described herein are not limited in this respect.

Additionally, or alternatively, in some embodiments, to identify the updated plurality of variants, the variant identification module 256 may be configured to compare haplotypes of the child to the haplotypes of each of the biological parents to identify candidate Mendelian violation loci. For example, the variant identification module 256 may use software configured to compare haplotypes of individuals using variants identified by variant calling software. A nonlimiting example of haplotype comparison software includes Real Time Genomics (RTG) vcfeval software. The RTG vcfeval software is described by Cleary, John G., et al. (“Comparing variant call files for performance benchmarking of next-generation sequencing variant calling pipelines.” BioRxiv (2015): 023754), which is incorporated by reference herein in its entirety.

Additionally, or alternatively, in some embodiments, to identify the updated plurality of variants, the variant identification module 256 may be configured to identify Mendelian violations. For example, the variant identification module 256 may use Mendelian violation identification software configured to identify Mendelian violations. A nonlimiting example of Mendelian violation identification software includes Real Time Genomics (RTG) Mendelian software may be used to identify the Mendelian violations. The RTG Mendelian software is described by Cleary, John G., et al. (“Comparing variant call files for performance benchmarking of next-generation sequencing variant calling pipelines.” BioRxiv (2015): 023754), which is incorporated by reference herein in its entirety.

In alternative embodiments, to identify the updated plurality of variants, the variant identification module 256 is configured to joint genotype the members of the family trio. The joint genotyping may be performed using results of aligning the sequence reads obtained for members of the family trio to the family genomic reference graph. For example, the variant identification module 256 may obtain from the sequence alignment module 252, results of aligning the sequence reads obtained from the family trio to a family genomic reference graph. For example, the variant identification module 256 may account for variant information across all members of the family trio, and output, for each member of the family trio, the most probable set of variants for that individual. In some embodiments, the variant identification module 256 may use joint genotyping software to perform the joint genotyping such as, for example, the Genome Analysis Toolkit (GATK) 3.0 software and GLnexus software.

Additionally, or alternatively, in some embodiments, the variant identification module 256 may be configured to identify one or more de novo variants from among the updated plurality of variants. In some embodiments, identifying the de novo variants includes comparing the sets of variants identified for the members of the family trio to identify variants identified for the child that were not identified for either of the biological parents.

In some embodiments, the graph generation module 254 obtains one or more genomic references (e.g., a linear genomic reference) from the genomic data store 280 and/or user(s) 290 (e.g., by user(s) uploading the genomic reference(s)). In some embodiments, the graph generation module 254 obtains variants from the variant identification module 256, genomic data store 280, and/or user(s) 290 (e.g., by the user(s) uploading the variants).

In some embodiments, the graph generation module 254 is configured to generate one or more genomic reference graphs. In some embodiments, generating a genomic reference graph includes augmenting a linear genomic reference with one or more variants (e.g., common among the global population, common among specific population(s) and/or identified for specific individuals). In some embodiments, this may be achieved by generating one or more data structures having node elements and edge elements that represent the linear genomic reference, and augmenting the data structure with node elements and edge elements that represent variants of the linear genomic reference. A node element may be represented as an object, and an object may store a pointer that represents an edge. Example techniques for generating a genomic reference graph are described by Rakocevic, G., et al. (“Fast and accurate genomic analysis using genome graphs.” Nat Genet. 51.2 (2019): 354-362).

In some embodiments, the graph generation module 254 is configured to generate a population-specific genomic reference graph. For example, in some embodiments, the graph generation module 254 may generate a genomic reference graph that represents a linear genomic reference and variants that are common to one or more specific populations. For example, the specific populations may include those to which the members of the family trio belong. Example techniques for generating a population-specific genomic reference graph are described by Tetikol, H. S., et al. (“Pan-African genome demonstrates how population-specific genome graphs improve high-throughput sequencing data analysis.” Nature Communications 13.1 (2022): 4384).

In some embodiments, the graph generation module 254 is configured to generate a family genomic reference graph that is specific to the members of the family trio. For example, the graph generation module 254 may be configured to augment a linear genomic reference with variants that have been identified for the members of the family trio. For example, in some embodiments, the graph generation module 254 may obtain variants from variant identification module 256 that were identified as a result of aligning sequence reads for members of the family trio to an initial genomic reference (e.g., a linear genomic reference, a population-specific genomic reference graph, etc.), and augment a linear genomic reference using the identified variants.

In some embodiments, the graph generation module 254 is further configured to process the variants identified for the family trio, prior to using them to generate a family genomic reference graph. For example, the graph generation module 254 may be configured to normalize the variants, filter the variants, and/or merge the variants. In some embodiments, the graph generation module 254 is configured to use variant processing software to process the variants. For example, the graph generation module 254 may use BCFtools, which is described by Li H. (“A statistical framework for SNP calling, mutation discovery, association mapping and population genetical parameter estimation from sequencing data.” Bioinformatics (2011) 27 (21) 2987-93), which is incorporated by reference herein in its entirety. Example techniques for processing variants are described herein including at least with respect to act 156 shown in FIG. 1B.

In some embodiments, the filtering module 258 is configured to obtain variants from variant identification module 256, user(s) 290 (e.g., by uploading variants), and/or genomic data store 280. The obtained variants may include one or more sets of variants such as a set of variant for a child and sets of variants for biological parents of the child. Additionally, or alternatively, the obtained variants may include a merged set of variants representing variants present in multiple members of the family trio.

In some embodiments, the filtering module 258 is configured to filter the obtained variants. For example, the variants may be filtered based metrics indicative of variant quality. Nonlimiting examples of variant quality metrics include quality by depth (QD), genotype quality (GQ), variant depth, allelic balance (AB), and mapped allele depth (MAD). In some embodiments, the filtering module 258 is configured to use filtering techniques to filter the obtained variants. Example filtering techniques are further described herein including at least with respect to act 364 shown in FIG. 3C.

In some embodiments, the disease identification module 264 may obtain variants and/or variant information from the variant identification module 256, the genomic data store 280, and user(s) 290 (e.g., by uploading the variants and/or the information about the variants). For example, the variants may include one or more variants identified as de novo variants and/or one or more variants identified as inherited variants. The variant information may include any suitable information about the variants such as, for example, an indication of whether a particular variant is a de novo or inherited variant, an indication as to which parent a variant was inherited from, and/or a genomic position of the variant.

In some embodiments, the disease identification module 264 may identify a disease associated with one or more variants identified for one or more members of the family trio. For example, the disease identification 264 may identify a disease associated with a de novo variant identified for the child of the family trio. In some embodiments, the disease identification module 264 may obtain information about diseases associated with particular variants and use the information to identify the disease for the member of the family trio. For example, the disease identification module 264 may obtain information about disease(s) and associated variants from the genomic data store 280, or from any other suitable source(s), as aspects of the technology described herein are not limited in this respect.

In some embodiments, software 250 further includes user interface module 262. User interface module 262 may be configured to generate a graphical user interface through which a user may provide input and view information generated by software 250. For example, in some embodiments, the user interface module 262 may be a webpage or web application accessible through an Internet browser. In some embodiments, the user interface module 262 may generate a graphical user interface (GUI) of an app executing on the user's mobile device. In some embodiments, the user interface module 262 may generate a GUI on a sequencing platform, such as sequencing platform 270. In some embodiments, the user interface module 262 may generate a number of selectable elements through which a user may interact. For example, the user interface module 262 may generate dropdown lists, checkboxes, text fields, or any other suitable element.

In some embodiments, the user interface module 262 is configured to generate a GUI including one or more results of processing sequencing reads obtained from the family trio. For example, the GUI may include an indication of one or more variants identified for each of one or more members of the family trio. Additionally, or alternatively, in some embodiments, the GUI may include an indication of one or more diseases identified for one or more members of the family trio. Additionally, or alternatively, in some embodiments, the GUI may include results of aligning sequence reads to a genomic reference (e.g., aligned positions of sequence reads, quality of alignment, etc.). It should be appreciated that the GUI may include any other suitable information, displayed in any suitable manner, as aspects of the technology described herein are not limited in this respect.

As shown in FIG. 2, system 200 also includes sequencing platform 270. In some embodiments, sequence reads are obtained from the sequencing platform 270. For example, the sequence alignment module 252 may obtain (either pull or be provided) the sequence reads from the sequencing platform 270. The sequencing platform 270 may be one of any suitable type such as, for example, any of the sequencing platforms described herein including at least with respect to FIG. 1A and with respect to the section “Sequencing Data.”

System 200 further includes genomic data store 280. In some embodiments, the genomic data store 280 stores sequence reads that were previously-obtained for one or more subjects (e.g., members of the family trio). Additionally, or alternatively, genomic data store 280 stores one or more genomic references (e.g., linear genomic reference(s) and/or genomic reference graph(s)). Additionally, or alternatively, genomic data store 280 stores variants previously-identified for one or more subjects (e.g., members of the family trio) and/or variants output at one or various stages of processing (e.g., variants output by variant identification module 256, variants output by filtering module 258, etc.). Additionally, or alternatively, genomic data store 280 may store variant information. Additionally, or alternatively, genomic data store 280 may store information about diseases associated with different variants. It should be appreciated that the genomic data store 280 may store any other suitable type of information, as aspects of the technology are not limited in this respect.

The genomic data store 280 may be of any suitable type (e.g., database system, multi-file, flat file, etc.) and may store genomic data in any suitable way in any suitable format, as aspects of the technology described herein are not limited in this respect. The genomic data store 280 may be part of or external to the computing device(s) 210.

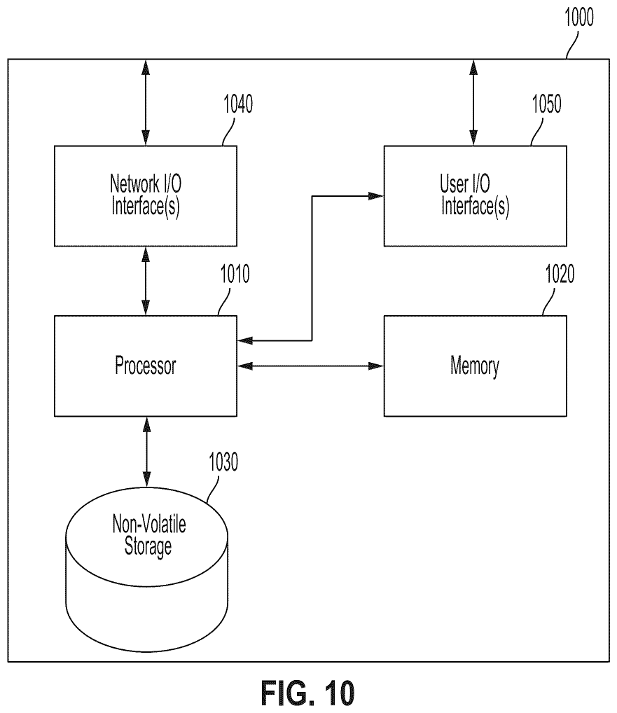

FIG. 3A is a flowchart of an illustrative process 300 for genotyping a family trio, according to some embodiments of the technology described herein. One or more acts (e.g., all acts) of process 300 may be performed automatically by any suitable computing device(s). For example, the act(s) may be performed by a laptop computer, a desktop computer, one or more servers, in a cloud computing environment, computing device 1000 as described herein with respect to FIG. 10, and/or in any other suitable way.

At act 302, sequence reads are obtained for one or more members of a family trio (e.g., a child and the biological parents of the child). In some embodiments, the sequence reads were previously-obtained by sequencing biological samples obtained from members of the family trio. For example, in some embodiments, the sequence reads were previously-obtained by sequencing germline samples obtained from members of the family trio. The germline samples may include blood samples, saliva samples, or any other suitable type of germline sample as aspects of the technology described herein are not limited in this respect. Examples of biological samples are described herein including at least with respect to FIG. 1A and with respect to the section “Biological Samples.”

In some embodiments, the sequence reads were previously-obtained using a sequencing platform such as a next generation sequencing platform (e.g., Illumina®, Roche®, Ion Torrent®, etc.), or any high-throughput or massively parallel sequencing platform. In some embodiments, these methods may be automated, in some embodiments, there may be manual intervention. In some embodiments, the sequence reads may be the result of non-next generation sequencing (e.g., Sanger sequencing). Examples of sequencing techniques are described herein including at least with respect to the section “Sequencing Data.”

In some embodiments, the sequence reads are obtained, at act 302, from a sequencing platform (e.g., sequencing platform 270 shown in FIG. 2), a data store (e.g., genomic data store 280 shown in FIG. 2), from one or more user(s) of the computing device used to implement process 300 (e.g., by uploading the sequence reads), or from any other suitable source, as aspects of the technology described herein are not limited in this respect.

In some embodiments, the sequence reads obtained at act 302 may include a set of sequence reads obtained for a child of the family trio, a set of sequence reads obtained for one biological parent of the child (e.g., the mother), and a set of sequence reads obtained for the other biological parent of the child (e.g., the father). In some embodiments, each set of sequence reads includes any suitable number of sequence reads such as, for example, at least 10,000 sequence reads, at least 100,000 sequence reads, at least 1,000,000 sequence reads, at least 10,000,000 sequence reads, at least 100,000,000 sequence reads, or any other suitable number of sequence reads, as aspects of the technology described herein are not limited in this respect.

In some embodiments, the sequence reads obtained at act 302 are in any suitable format. For example, the sequence reads may be specified in one or more files such as FASTQ files. For example, multiple FASTQ files may be obtained (e.g., one for each member of the family trio).

At act 304, the sequence reads obtained at act 302 are aligned to an initial genomic reference. The initial genomic reference may include any genomic reference suitable for genotyping a subject such as one or more members of family trio, as aspects of the technology described herein are not limited in this respect. For example, in some embodiments, the initial genomic reference includes a linear genomic reference. The linear genomic reference may include a linear human genome reference sequence such as, for example, human genome version 19 (hg19), hg38, Genome Reference Consortium human reference 38 (GRCh38), GRCh37, or any other suitable linear human genome reference sequence. In some embodiments, the linear genomic reference is stored in any suitable format such as, for example, FASTA file format.

In some embodiments, the initial genomic reference includes a genomic reference graph representing a linear reference sequence having nodes and edges and edges. The genomic reference graph may be one or more data structures that specify nodes and edges connecting the nodes. The nodes may represent nucleotide sequences stored as respective strings of one or more symbols, and the edges may represent a connection between at least two of the nodes. Alternatively, the edges may represent nucleotide sequences stored as respective strings of one or more symbols, and the nodes may represent a connection between at least two of the edges. In some embodiments, the data structure includes objects that represent the nodes and pointers that represent the edges. As one non-limiting example, the data structure may be a directed acyclic graph (DAG). As another non-limiting example, the data structure may be a directed graph with one or more cycles to represent repeats. Example techniques for generating a genomic reference graph are described by Rakocevic, G., et al. (“Fast and accurate genomic analysis using genome graphs.” Nat Genet. 51.2 (2019): 354-362).

Additionally, or alternatively, in some embodiments, the initial genomic reference includes a genomic reference graph representing a linear reference sequence and variation of the linear reference sequence. For example, such a genomic reference graph may be generated by representing a linear genomic reference as a graph having nodes and edges and augmenting the linear genomic reference with one or more nodes and one or more edges representing at least some variants.

In some embodiments, the initial genomic reference graph is specific to one or more populations. Such a reference graph may represent variants that common among members of the one or more populations. For example, the initial genomic reference graph may represent variants that are common among members of the one or more populations to which the members of the family trio belong. The population(s) to which the members of the family trio belong may be identified using any suitable techniques, as aspects of the technology are not limited in this respect. Example techniques for generating a population-specific genomic reference graph are described by Tetikol, H. S., et al. (“Pan-African genome demonstrates how population-specific genome graphs improve high-throughput sequencing data analysis.” Nature Communications 13.1 (2022): 4384).

In some embodiments, when the initial genomic reference is a linear genomic reference, sequence reads may be aligned to the linear genomic reference, at act 304, using any suitable linear alignment techniques, as aspects of the technology described herein are not limited in this respect. In some embodiments, the alignment may be performed using dynamic programming. Nonlimiting examples of linear alignment techniques include, but are not limited to, the Needleman-Wunsch algorithm, the Smith-Waterman algorithm, and Burrows-Wheeler Alignment (BWA), among others.

In some embodiments, when the initial genomic reference is a genomic reference graph representing a linear genomic reference and variation thereof, sequence reads may be aligned to the genomic reference graph, at act 304, using any suitable graph alignment techniques, as aspects of the technology described herein are not limited in this respect. In some embodiments, the graph alignment may be performed using dynamic programming. In some embodiments, one or more linear sequence alignment techniques may be modified to handle the branches and merges present in a genomic reference graph. Example graph alignment techniques are described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”. Example techniques for aligning sequence reads to a genomic reference graph are further described herein including at least with respect to FIG. 4B.

In some embodiments, one or more files are output as a result of aligning the sequence reads to the initial genomic reference. The file(s) may include information representing the aligned sequence reads with respect to the initial genomic reference. The file(s) may be in any suitable format for representing aligned sequences such as, for example, sequence alignment map (SAM) file format or binary alignment map (BAM) file format, or compressed reference-oriented alignment map (CRAM) file format. In some embodiments, a different file may be output for each member of the family trio.

At act 306, an initial plurality of variants is identified based on results of aligning the sequence reads to the initial genomic reference at act 304. In some embodiments, the initial plurality of variants includes an initial set of variants for the child of the family trio, an initial set of variants for one biological parent of the child (e.g., the mother), and an initial set of variants for the other biological parent of the child (e.g., the father). In some embodiments, identifying a set of variants for an individual includes identifying where the aligned sequence reads for that individual differs from the genomic reference. In some embodiments, this is performed using variant calling software. Nonlimiting examples of variant calling software include GRAF Variant Caller software, Genomic Atlas Toolkit (GATK) software, SAMtools software, BCFtools software, or any other suitable variant calling software as aspects of the technology described herein are not limited in this respect.

In some embodiments, the output of act 306 includes one or more files that include information indicative of the initial plurality of variants. For example, a different file may be obtained for each member of the family trio, each of which includes information indicative of an initial set of variants obtained for the particular member of the family trio. The file(s) may be in any suitable format such as, for example, Variant Call Format (VCF).

In some embodiments, the initial plurality of variants identified at act 306 are (optionally) processed, at act 308, prior to being used to generate the family genomic reference graph at act 310. In some embodiments, each set of the initial plurality of variants (e.g., the initial set of variants obtained for the child and the initial sets of variants obtained for the parents) is processed. In some embodiments, any suitable variant processing techniques may be used, as aspects of the technology are not limited in this respect. For example, in some embodiments, the processing may include normalizing the variants, filtering the variants, and/or merging the variants (e.g., merging the different sets of variants obtained for the different members of the family trio). In some embodiments, variant processing software may be used to process the variants. For example, BCFtools software may be used. BCFtools is described by Li H. (“A statistical framework for SNP calling, mutation discovery, association mapping and population genetical parameter estimation from sequencing data.” Bioinformatics (2011) 27 (21) 2987-93), which is incorporated by reference herein in its entirety. Example techniques for processing variants are described herein including at least with respect to act 156 shown in FIG. 1B and with respect to FIG. 4C.

At act 310, a family genomic reference graph is generated using the initial plurality of variants. In some embodiments, the family genomic reference graph is generated at least in part by augmenting a linear reference with the initial plurality of variants (e.g., the processed initial plurality of variants). For example, the linear reference may be represented by nodes connected by edges. The nodes may represent nucleotide sequences stored as respective strings of one or more symbols, and the edges may represent a connection between at least two of the nodes. Alternatively, the edges may represent nucleotide sequences stored as respective strings of one or more symbols, and the nodes may represent a connection between at least two of the edges. The linear reference may be augmented by including, at one or more positions along the linear reference, alternative nodes and/or edges, thereby generating alternative paths through a genomic graph reference. For example, node(s) may be used to represent an insertion at the position and an edge may be used to represent a deletion. Example techniques for generating a genomic reference graph are described by Rakocevic, G., et al. (“Fast and accurate genomic analysis using genome graphs.” Nat Genet. 51.2 (2019): 354-362)

At act 312, at least some (e.g., all) of the sequence reads obtained at act 302 are aligned to the family genomic reference graph. For example, sequence reads obtained for each member of the family trio may be aligned to the family genomic reference graph at act 312. In some embodiments, the sequence reads may be aligned to the family genomic reference graph using any suitable graph alignment techniques, as aspects of the technology described herein are not limited in this respect. In some embodiments, the graph alignment may be performed using dynamic programming. In some embodiments, one or more linear sequence alignment techniques may be modified to handle the branches and merges present in a genomic reference graph. Example graph alignment techniques are described by Rakocevic, G., et al. (“Fast and accurate genomic analysis using genome graphs.” Nat Genet. 51.2 (2019): 354-362) and in U.S. Pat. No. 9,116,866, entitled “METHODS AND SYSTEMS FOR DETECTING SEQUENCE VARIANTS”. Example techniques for aligning sequence reads to a genomic reference graph are further described herein including at least with respect to FIG. 4B.

In some embodiments, one or more files are output as a result of aligning the sequence reads to the family genomic reference graph. The file(s) may include information representing the aligned sequence reads with respect to the family genomic reference graph. The file(s) may be in any suitable format for representing aligned sequences such as, for example, sequence alignment map (SAM) file format or binary alignment map (BAM) file format, or compressed reference-oriented alignment map (CRAM) file format. In some embodiments, a different file may be output for each member of the family trio.

At act 314, an updated plurality of variants is identified based on results of aligning the sequence reads to the family genomic reference graph at act 312. In some embodiments, identifying the updated plurality of variants is performed using results of aligning the sequence reads obtained for members of the family trio to the family genomic reference graph. For example, identifying the updated plurality of variants may be performed using one or more files representing aligned sequence reads (e.g., files in SAM file format, BAM file format, CRAM file format, etc.) Example techniques for identifying an updated plurality of variants are described herein including at least with respect to process 320 shown in FIG. 3B and process 360 shown in FIG. 3C.

In some embodiments, the output of act 314 includes the updated plurality of variants. The updated plurality of variants may include an updated set of variants for the child, an updated set of variants for the biological mother of the child, and an updated set of variants for the biological father of the child. The updated plurality of variants may be output in any suitable format for representing variants such as, for example, variants call format (VCF).

At act 316, de novo variants are (optionally) identified from among the updated plurality of variants identified at act 314. For example, the de novo variants may be identified as variants that are included in the updated set of variants identified for the child but which are not included in the updated sets of variants identified for either of the biological parents of the family trio.

It should be appreciated that process 300 may include one or more additional or alternative acts not shown in FIG. 3A. For example, process 300 may include an act for sequencing the biological samples obtained from the members of the family trio to obtain the sequence reads. Additionally, or alternatively, process 300 may include an act for using the de novo variants to identify a disease associated with one or more members of the family trio.

FIG. 3B is a flowchart of an illustrative process 320 for identifying an updated plurality of variants, according to some embodiments of the technology described herein. One or more acts (e.g., all acts) of process 320 may be performed automatically by any suitable computing device(s). For example, the act(s) may be performed by a laptop computer, a desktop computer, one or more servers, in a cloud computing environment, computing device 1000 as described herein with respect to FIG. 10, and/or in any other suitable way.

At act 322, an intermediate plurality of variants is identified for the family trio based on results of aligning sequence reads to a genomic reference (e.g., aligning sequence reads to the family genomic reference graph at act 312 of process 300 shown in FIG. 3A). In some embodiments, the intermediate plurality of variants includes a first intermediate set of variants for the first biological parent, a second intermediate set of variants for the second biological parent, and a third intermediate set of variants for the child. In some embodiments, identifying an intermediate set of variants for a member of the family trio includes identifying variants based on the alignment of the sequence reads obtained for the member of the family (e.g., sequence reads obtained at act 302 of process 300 in FIG. 3A) to the family genomic reference graph at act 312 of process 300 shown in FIG. 3A. For example, identifying the intermediate set of variants may include identifying where the aligned sequence reads for that individual differs from the family genomic reference graph. In some embodiments, this is performed using variant calling software. Nonlimiting examples of variant calling software include GRAF Variant Caller software, Genomic Atlas Toolkit (GATK) software, SAMtools software, BCFtools software, or any other suitable variant calling software as aspects of the technology described herein are not limited in this respect.

At act 324, the intermediate plurality of variants is filtered. In some embodiments, the filtering of a variant is based on a metric indicative of a confidence associated with the variant. For example, a variant with a metric value that is less than a threshold may be filtered out, while a variant with a metric value that is greater than or equal to the threshold may be included in a filtered set of variants and used downstream for further analysis. Nonlimiting examples of metrics indicative of confidence include quality by depth (QD) and genotype quality (GQ).

Quality by depth (QD) refers to genotype quality (e.g., variant quality) normalized by read depth. Genotype quality refers to a value indicative of the confidence that there is a variation at a given aligned position (e.g., a position at which sequence read(s) are aligned to a genomic reference, such as a family genomic reference graph). In some embodiments, QD is output as a result of performing variant identification (e.g., at act 322). For example, the QD may be output by variant identification software. In some embodiments, filtering a variant based on its QD includes determining whether its QD is greater than or equal to a QD threshold, and filtering out the variant (excluding it from further analysis) if its QD is less than the threshold. The QD threshold may be any suitable threshold as aspects of the technology described herein are not limited in this respect. For example, the QD threshold may be between 0.5 and 5, between 0.6 and 4, between 0.7 and 3, between 0.8 and 2, between 0.9 and 1, or within any other suitable range. Additionally, or alternatively, the QD threshold may be, at least 0.5, at least 0.6, at least 0.7, at least 0.8, at least 0.9, at least 1, at least 2, at least 3, at least 4 at least 5, or at least any other suitable value. Additionally, or alternatively, the QD threshold may be at most 10, at most 8, at most 6, at most 5, at most 4, at most 3, at most 2, or at most 1, or at most any other suitable value. It should be appreciated that any of the above-listed upper bounds may be coupled with any of the above-listed lower bounds.

In some embodiments, genotype quality (GQ) is output as a result of performing variant identification (e.g., at act 322). For example, the GQ may be output by variant identification software. In some embodiments, filtering a variant based on its GQ includes determining whether its GQ is greater than or equal to a GQ threshold, and filtering out the variant (excluding it from further analysis) if its GQ is less than the threshold. The GQ threshold may be any suitable threshold as aspects of the technology described herein are not limited in this respect. For example, the GQ threshold may be between 5 and 35, between 10 and 30, between 15 and 25, between 18 and 22, or between any other suitable range. Additionally, or alternatively, the GQ threshold may be at least one, at least 5, at least 10, at least 15, at least 18, at least 20, at least 22, at least 25, at least 35, at least 40, at least 50, or any other suitable value. Additionally, or alternatively, the GQ threshold may be at most 10, at most 15, at most 20, at most 22, at most 25, at most 30, at most 25, at most 40, at most 50, or at most any other suitable value. It should be appreciated that any of the above-listed upper bounds may be coupled with any of the above-listed lower bounds.

In some embodiments, the filtered variants are used to identify differences between haplotypes of the child and haplotypes of each of the biological parents. For example, at act 324, first differences are identified between haplotypes of the child and haplotypes of the first biological parent using the first intermediate set of variants and the third intermediate set of variants. At act 326, second differences are identified between the haplotypes of the child and haplotypes of the second biological parent using the second intermediate set of variants and the third intermediate set of variants. In some embodiments, differences are identified between haplotypes using software configured to compare haplotypes of different individuals. Any suitable haplotype comparison software may be used, as aspects of the technology described herein are not limited in this respect. As one non-limiting example, the Real Time Genomics (RTG) vcfeval software may be used to compare haplotypes of different members of the family trio.

At act 330, one or more candidate Mendelian violation loci are identified based on the first differences between the haplotypes of the child and the first parent and the second differences between the haplotypes of the child and the second parent. A candidate Mendelian violation locus may refer to a region in the child's genome where the child's haplotypes differ from both the haplotypes of the first biological parent and the haplotypes of the second biological parent. The candidate Mendelian violation loci may be identified by identifying loci for which the first differences and the second differences each indicate a difference.

At act 332, the intermediate plurality of variants is filtered based on the one or more candidate Mendelian violation loci. In some embodiments, the filtering includes filtering by region. For example, variants that do not correspond to the candidate Mendelian violation loci may be filtered out. Variants that are filtered out may correspond to inherited variants and therefore should not violate Mendelian constraints. In some embodiments, the filtering may be performed using any suitable software configured to filter out variants by region, as aspects of the technology described herein are not limited in this respect. Example software for filtering variants by region is described by Danecek, Petr, et al. (“Twelve years of SAMtools and BCFtools.” Gigascience 10.2 (2021): giab008), which is incorporated by reference herein in its entirety.

In some embodiments, prior to being filtered at act 332, the intermediate sets of variants (e.g., the first intermediate set, the second intermediate set, and the third intermediate set) are merged. The sets of variants may be merged using any suitable techniques, as aspects of the technology described herein are not limited in this respect. For example, the sets of variants may be merged using software configured to perform the merging. As a non-limiting example, BCF tools software may be used to merge the variants.

At act 334, one or more Mendelian violations are identified using the filtered, intermediate plurality of variants obtained at act 332. In some embodiments, the Mendelian violations include variants that were identified in the genome of the child, but not in the genome of either of the parents. The Mendelian violations may be de novo variants or may be the result of an error (e.g., a sequencing error). In some embodiments, the one or more Mendelian violations may be identified using any suitable software configured to identify Mendelian violations, as aspects of the technology described herein are not limited in this respect. As one non-limiting example, the Real Time Genomics (RTG) Mendelian software may be used to identify the Mendelian violations.

At act 336, the one or more Mendelian violations are filtered to identify one or more de novo variants for the subject. In some embodiments, filtering the Mendelian violations may include filtering based on coverage. Filtering a Mendelian violation based on coverage may include, for each member of the family trio, (a) determining the proportion of mapped sequence reads supporting the allele at the position of the Mendelian violation, (b) and comparing the determined proportion to a threshold to determine whether the proportion is less than the threshold. If the determined proportion for any the family trio members is less than the threshold, then the Mendelian violation is excluded. This may indicate that the Mendelian violation is the result of an error (e.g., a sequencing error). If the determined proportions are greater than or equal to the threshold, the Mendelian violation may be identified as a de novo variant for the child and not filtered out.

In some embodiments, filtering the Mendelian violations may also include filtering based on allelic balance (AB). For example, a Mendelian violation may be filtered out when any allele at the location of the violation has an AB value less than a first specified threshold (e.g., 0.05, 0.10, 0.15, 0.2, 0.25, 0.3 any threshold in the range of 0.01 and 0.3) and/or when the sum of AB values for the alleles at the violation location is less than a second specified threshold (e.g., 0.75, 0.8, 0.85, 0.90, 0.95, any threshold in the range of 0.75 and 0.99). Allelic balance, for an allele, refers to the proportion of sequence reads supporting the allele. The proportion may be calculated as a ratio of the number sequence reads supporting the allele (e.g., using the allele depth value reporting by the variant caller (VCF) or counting the number of sequence reads aligned to the allele in the BAM file) to the total number of sequence reads aligned to the position of the violation.”

Example filtering techniques are described by Danecek, Petr, et al. (“Twelve years of SAMtools and BCFtools.” Gigascience 10.2 (2021): giab008), which is incorporated by reference herein in its entirety.

In some embodiments, the output of act 336 includes one or more de novo variants for the child. In some embodiments, the de novo variants may be included in an updated plurality of variants that is provided as output. For example, the updated plurality of variants may include both the de novo variants, as well one or more inherited variants. Inherited variants may represent variants that are shared by at least two members of the family trio.

FIG. 3C is a flowchart of an illustrative process 360 for identifying an updated plurality of variants, according to some embodiments of the technology described herein. One or more acts (e.g., all acts) of process 360 may be performed automatically by any suitable computing device(s). For example, the act(s) may be performed by a laptop computer, a desktop computer, one or more servers, in a cloud computing environment, computing device 1000 as described herein with respect to FIG. 10, and/or in any other suitable way.

At act 362, the members of the family trio may be joint genotyped based on results of aligning sequence reads to the family genomic reference graph (e.g., at act 312 of process 300 shown in FIG. 3A). Joint genotyping refers to the process of (a) independently identifying potential variants for each member in the family trio based on the aligned positions of the individual's sequence reads relative to the family genomic reference graph, and (b) using statistical techniques to refine the potential variants identified for each member of the family trio by considering the potential variants identified for the other members of the family trio. By sharing information across the members of the family trio, joint genotyping allows for the identification of variants in one or more of the members that might have otherwise been filtered out due to poor coverage of the variant and/or poor quality of the sequence reads. Consider, for example, a variant that is identified for one of the biological parents of the family trio, but which has low coverage (e.g., a coverage below a threshold coverage). If the variant is identified for the child of the family trio, with high coverage (e.g., coverage above the coverage threshold), then it may be inferred with joint genotyping that the variant should be identified for the biological parent and should not be filtered out due to low coverage. Accordingly, joint genotyping allows the variant to be accurately identified as a variant that has been inherited by the child from the parent, as opposed to being inaccurately identified as a de novo variant for the child.

In some embodiments, joint genotyping is performed using joint genotyping software such as, for example, the Genome Analysis Toolkit (GATK) 3.0 and GLnexus. Joint genotyping using the GATK 3.0 software is described by Poplin R, et al. (“Scaling accurate genetic variant discovery to tens of thousands of samples.” BioRxiv (2017): 201178), which is incorporated by reference herein in its entirety. GLnexus is described by Lin, M. F., et al. (“GLnexus: joint variant calling for large cohort sequencing.” BioRxiv (2018): 343970), which is incorporated by reference herein in its entirety.

At act 364, the variants identified by joint genotyping the members of the family trio may be filtered to obtain the family trio variants 108. The variants may be filtered based on metric(s) indicative of the quality of the variant. For example, the metric(s) may be compared to criteria used for determining whether a particular variant should be filtered out (excluded from further analysis). Nonlimiting examples of metrics indicative of variant quality include quality by depth (QD), genotype quality (GQ), depth, allelic balance (AB), and mapped allele depth (MAD).

Quality by depth (QD) and genotype quality (GQ) are described herein including at least with respect to act 324 of process 320 shown in FIG. 3B.

Depth refers to the total number of sequence reads aligned to the variant position. In some embodiments, depth is output as a result of performing joint genotyping. For example, the depth may be output by joint genotyping software. In some embodiments, filtering a variant based on depth includes comparing its depth to respective depth criteria, and filtering out the variant if its depth does not satisfy the respective depth criteria. In some embodiments, the depth criteria depend on the type of sequencing that was used to obtain the sequence reads used to identify for the variant. For example, different depth criteria may be used for filtering variants identified using WGS sequence reads and variants identified using WES sequence reads.

In some embodiments, for variants identified using WGS sequence reads, the depth criteria may include a range of percentiles, and filtering the variant based on depth may include determining whether the depth falls within the range of percentiles and filtering out the variant if it does not fall within the range. The range of percentiles may be based on the distribution of depths determined for variants identified for an individual (e.g., a member of the family trio). The range of percentiles may be any suitable range as aspects of the technology described herein are not limited in this respect. For example, the range of percentiles may be between the 2nd percentile and the 98^{th} percentile, between the 5^{th} percentile and the 97^{th} percentile, between the 6^{th} percentile and the 96^{th} percentile, between the 7^{th} percentile and the 95^{th} percentile, between the 8^{th} percentile and the 94^{th} percentile, between the 9^{th} percentile and the 92^{nd} percentile, between the 10^{th} percentile and the 90^{th} percentile, between the 25^{th} percentile and the 75th percentile, or any other suitable range of percentiles. Additionally, or alternatively, the upper bound of the range of percentiles may be at most the 98^{th} percentile, at most the 97^{th} percentile, at most the 96^{th} percentile, at most the 95^{th} percentile, at most the 94^{th} percentile, at most the 92^{nd} percentile, at most the 90^{th} percentile, at most the 75^{th} percentile or any other suitable upper bound. Additionally, or alternatively, the lower bound of the range of the percentiles may be at least the 2^{nd} percentile, at least the 5^{th} percentile, at least the 6^{th} percentile, at least the 7th percentile, at least the 8^{th} percentile, at least the 9^{th} percentile, at least the 10^{th} percentile, at least the 25^{th} percentile, or any other suitable lower bound, as aspects of the technology described herein are not limited in this respect. It should be appreciated that any of the above-listed upper bounds may be coupled with any of the above-listed lower bounds.

In some embodiments, for variants identified using WES sequence reads, the depth criteria may include a threshold depth, and filtering the variants based on depth may include determining whether its depth is greater than or equal to threshold depth, and filtering out the variant if its depth is not greater than or equal to the threshold depth. The threshold depth may be any suitable threshold depth, as aspects of the technology described herein are not limited in this respect. For example, the threshold depth may be between 2 and 20, between 3 and 18, between 4 and 15, between 5 and 10, between 6 and 8, or within any other suitable range. Additionally, or alternatively, the threshold depth may be at least 2, at least 3, at least 4, at least 5, at least 6, at least 7, at least 8, at least 9, at least 10, at least 11, at least 12, at least 15, at least 20, or at least any other suitable threshold depth. Additionally, or alternatively, the threshold depth may be at most 5, at most 6, at most 7, at most 8, at most 9, at most 10, at most 11, at most 12, at most 15, at most 20, or at most any other suitable threshold depth. It should be appreciated that any of the above-listed upper bounds may be coupled with any of the above-listed lower bounds. In some embodiments, the threshold depth may depend on the individual for whom the variant was identified. For example, a different threshold depth may be used to filter variants identified for biological parents (e.g., a threshold depth of 5) than the threshold depth used to filter variants identified for the child of the family trio (e.g., a threshold depth of 10).

Allelic balance (AB) refers to the ratio of sequence reads supporting the mapped allele (e.g., second most common allele in the family trio) to the depth (e.g., the total number of sequence reads aligned to the variant position). In some embodiments, AB is output as a result of performing joint genotyping. For example, the AB may be output by joint genotyping software. In some embodiments, filtering a variant based on AB includes comparing its AB to respective AB criteria, and filtering out the variant if its AB does not satisfy the respective AB criteria. In some embodiments, the AB criteria depends on the individual for whom the variant was identified. For example, different AB criteria may be used to filter variants obtained for biological parents of the family trio than the AB criteria used to filter variants obtained for the child of the family trio.

In some embodiments, for a variant obtained for the biological parents of the family trio, the AB criteria may include a threshold AB, and filtering the variant may include determining whether its AB is greater than or equal to the threshold AB, and filtering out the variant if its AB is not greater than or equal to the threshold AB. The threshold AB may be any suitable threshold AB, as aspects of the technology described herein are not limited in this respect. For example, the threshold AB may be between 0.01 and 0.2, between 0.02 and 0.15, between 0.03 and 0.1, between 0.04 and 0.08, or within any other suitable range. Additionally, or alternatively, the threshold AB may be at least 0.01, at least 0.02, at least 0.03, at least 0.04, at least 0.05, at least 0.06, at least 0.07, at least 0.08, at least 0.09, at least 0.10, or at least any other suitable value. Additionally, or alternatively, the threshold AB may be at most 0.04, at most 0.05, at most 0.06, at most 0.07, at most 0.08, at most 0.09, at most 0.10, at most 0.15, at most 0.18, at most 0.2, or at most any other suitable value. It should be appreciated that any of the above-listed upper bounds may be coupled with any of the above-listed lower bounds.

In some embodiments, for a variant obtained for the child of the family trio, the AB criteria may include a pre-determined range, and filtering the variant based on its AB may include determining whether its AB is within the pre-determined range, and filtering out the variant if its AB is not within the pre-determined range. The pre-determined range maybe any suitable range as aspects of the embodiments described herein are not limited in this respect. For example, the pre-determined range may be a range between 0.05 and 0.95, a range between 0.10 and 0.90, a range between 0.15 and 0.8, a range between 0.20 and 0.89, a range between 0.30 and 0.88, a range between 0.40 and 0.87, a range between 0.50 and 0.86, a range between 0.60 and 0.85, a range between 0.70 and 0.84, a range between 0.75 and 0.83, or any other suitable range. Additionally, or alternatively, the upper bound of the range may be at most 0.98, at most 0.95, at most 0.90, at most 0.89, at most 0.88, at most 0.87, at most 0.86, at most 0.85, at most 0.84, at most 0.83, at most 0.80, at most 0.75, at most 0.70, or any other suitable upper bound. Additionally, or alternatively, the lower bound of the range may be at least 0.05, at least 0.10, at least 0.20, at least 0.30, at least 0.40, at least 0.50, at least 0.60, at least 0.70, at least 0.80, at least 0.85, or any other suitable lower bound. It should be appreciated that any of the above-listed upper bounds may be coupled with any of the above-listed lower bounds.

Mapped allele depth (MAD) refers to the number of sequence reads aligned to the minor allele (e.g., second most common allele in the family trio). In some embodiments, MAD is output as a result of performing joint genotyping. For example, the MAD may be output by joint genotyping software. In some embodiments, filtering a variant based on MAD includes comparing its MAD to respective MAD criteria, and filtering out the variant if its MAD does not satisfy the respective MAD criteria. In some embodiments, the MAD criteria depends on the individual for whom the variant was identified. For example, different MAD criteria may be used to filter variants obtained for biological parents of the family trio than the MAD criteria used to filter variants obtained for the child of the family trio.

In some embodiments, for a variant obtained for the biological parents of the family trio, the MAD criteria may include a threshold MAD, and filtering the variant may include determining whether its MAD is greater than or equal to the threshold MAD, and filtering out the variant if its MAD is not greater than or equal to the threshold MAD. The threshold MAD may be any suitable threshold MAD, as aspects of the technology described herein are not limited in this respect. For example, the threshold MAD may be between 1 and 10, between 2 and 9, between 3 and 8, between 4 and 7, or within any other suitable range. Additionally, or alternatively, the threshold MAD may be at least 1, at least 2, at least 3, at least 4, at least 5, at least 6, at least 7, at least 8, at least 9, at least 10, or any other suitable value. Additionally, or alternatively, the threshold MAD may be at most 2, at most 3, at most 4, at most 5, at most 6, at most 7, at most 8, at most 9, at most 10, at most 11, or any other suitable value. It should be appreciated that any of the above-listed upper bounds may be coupled with any of the above-listed lower bounds.

In some embodiments, for a variant obtained for the child of the family trio, the MAD criteria may include a threshold MAD, and filtering the variant may include determining whether its MAD is lesser than or equal to the threshold MAD, and filtering out the variant if its MAD is not lesser than or equal to the threshold MAD. The threshold MAD may be any suitable threshold MAD, as aspects of the technology described herein are not limited in this respect. For example, the threshold MAD may be between 1 and 10, between 2 and 9, between 3 and 8, between 4 and 7, or within any other suitable range. Additionally, or alternatively, the threshold MAD may be at least 1, at least 2, at least 3, at least 4, at least 5, at least 6, at least 7, at least 8, at least 9, at least 10, or any other suitable value. Additionally, or alternatively, the threshold MAD may be at most 2, at most 3, at most 4, at most 5, at most 6, at most 7, at most 8, at most 9, at most 10, at most 11, or any other suitable value. It should be appreciated that any of the above-listed upper bounds may be coupled with any of the above-listed lower bounds.

In some embodiments, the output of act 364 includes an updated plurality of variants for the family trio. The updated plurality of variants may include one or more de novo variants and/or one or more inherited variants.

FIG. 4A is an illustrative example 400 of genotyping a family trio, according to some embodiments of the technology described herein. As shown in FIG. 4A, the family trio includes a first biological parent 402-1, a second biological parent 402401-3, and a child 401-2 of the biological parent 401-1 and biological parent 401-3.

In the example 400, sequence reads are obtained for each of the members of the family trio. For example, sequence reads 402-1 are obtained from the first parent 401-1, sequence reads 402-3 are obtained from the second parent 401-3, and sequence reads 402-2 are obtained from the child 401-2. Example techniques for obtaining sequence reads from members of a family trio are described herein including at least with respect to act 302 of process 300 shown in FIG. 3A.

In the example 400, the obtained sequence reads are aligned to an initial genomic reference at act 403 to obtain aligned reads for each member of the family trio. The aligned reads include aligned reads 404-1 for the first parent 401-1, aligned reads 404-2 for the child 401-2, and aligned reads 404-3 for the second parent 401-3. Example techniques for aligning sequence reads to an initial genomic reference are described herein including at least with respect to act 304 of process 300 shown in FIG. 3A and example 50 shown in FIG. 4D.

In some embodiments, the aligned reads are used to identify an initial plurality of variants for the family trio at act 405. The initial plurality of variants may include an initial set of variants 406-1 for the first parent 401-1, an initial set of variants 406-3 for the second parent 401-3, and an initial set of variants 406-2 for the child 401-2. Example techniques for identifying an initial plurality of variants are described herein including at least with respect to act 306 of process 300 shown in FIG. 3A and example 450 shown in FIG. 4D.

In the example 400, the initial plurality of variants, including the initial set of variants 406-1, the initial set of variants 406-2, and the initial set of variants 406-3, are used to generate the family genomic reference graph at act 408. Example techniques for generating a family genomic reference graph are described herein including at least with respect to act 310 of process 300 shown in FIG. 3A and with respect to the example 470 shown in FIG. 4E.

In the example 400, at least some of the sequence reads obtained for the members of the family trio are aligned to family genomic reference graph generated at act 408. For example, at least some of the sequence reads 402-1 obtained for the first parent 401-1 may be aligned to the family genomic reference graph at act 409 to obtain aligned reads 410-1. At least some of the sequence reads 402-3 obtained for the second parent 401-3 may be aligned to the family genomic reference graph at act 409 to obtain aligned reads 410-3. At least some of the sequence reads 402-2 obtained for the child 401-2 may be aligned to the family genomic reference graph at act 409 to obtain aligned reads 410-2. Example techniques for aligning sequence reads to a family genomic reference graph are described herein including at least with respect to act 312 of process 300 shown in FIG. 3A and with respect to example 450 shown in FIG. 4D.

In the example 400, the aligned sequence reads 410-1, aligned sequence reads 410-2, and aligned sequence reads 410-3 may be used to identify an updated plurality of variants for the family trio at act 411. The updated plurality of variants 412 includes at least some de novo variants 413 (e.g., variants only identified for child 401-2) and at least some inherited variants 414 (e.g., variants identified for the child 401-2 and at least one or both of the biological parents). In some embodiments, the de novo variants 413 are identified from among the updated plurality of variants 412. Example techniques for identifying de novo variants from among an updated plurality of variants are described herein including at least with respect to act 316 of process 300 shown in FIG. 3A.

The example 400 further includes identifying a disease, at act 415, based on the de novo variants 413. For example, the disease may be associated with the de novo variants 413. The disease may be identified for the child 401-2 whose genome includes the de novo variants 413.

FIG. 4B is an illustrative example of identifying variants based on a result of aligning sequence reads to a family genomic reference graph, according to some embodiments of the technology described herein.

As shown in FIG. 4B, variants (e.g., intermediate variants) are identified at act 421 using aligned reads 410-1, aligned reads 410-2, and aligned reads 410-3. The aligned reads may include sequence reads that have been aligned to a genomic reference. For example, the aligned reads may include the sequence reads that were aligned to the family genomic reference graph at act 409 of example 400 shown in FIG. 4A. The aligned reads may be in any suitable format such as Binary Alignment Map (BAM) format or Sequence Alignment Map (SAM), as aspects of the technology described herein are not limited in this respect.

In some embodiments, the aligned sequence reads are used to identify variants for the family trio at act 421. Example techniques for identifying variants are described herein including at least with respect to act 322 of process 320 shown in FIG. 3B.

In some embodiments, the variants identified at act 421 are filtered at act 422 to obtain filtered variants. For example, aligned reads 410-1 may be used to identify variants for the first biological parent, and the identified variants may be filtered to obtain filtered variants 423-1 for the first biological parent. Aligned reads 410-2 may be used to identify variants for the child, and the identified variants may be filtered to obtain filtered variants 423-2 for the child. Aligned reads 410-3 may be used to identify variants for the second biological parent, and the identified variants may be filtered to obtain filtered variants 423-3 for the second biological parent. Example filtering techniques are described herein including at least with respect to act 324 of process 320 shown in FIG. 3B.

In the example 420, at act 424-1, the haplotypes of the child may be compared to the haplotypes of the first biological parent to identify differences 425-1 between the haplotypes. The comparison may be performed using the variants 423-1 identified for the first biological parent and the variants 423-2 identified for the child. At act 424-2, the haplotypes of the child may be compared to the haplotypes of the second biological parent to identify differences 425-2 between the haplotypes. The comparison may be performed using the variants 423-3 identified for the child and variants 423-3 identified for the second biological parent 423-3. Example techniques for identifying differences between the haplotypes of different individuals are described herein including at least with respect to act 326 and act 328 of process 320 shown in FIG. 3B.

The differences 425-1 between the haplotypes of the child and the first biological parent and the differences 425-2 between the haplotypes of the child and the second biological parent may be used to identify candidate Mendelian violation loci 427 at act 426. As described herein, including at least with respect to act 330, a candidate Mendelian violation locus may refer to a region in the child's genome where the child's haplotypes differ from both the haplotypes of the first biological parent and the haplotypes of the second biological parent. The candidate Mendelian violation loci may be identified by identifying loci for which the first differences 425-1 and the second differences 425-2 each indicate a difference.

As shown in example 420, the variants 423-1, variants 423-2, and variants 423-3 may be merged at act 433 to obtain merged variants 434. The variants may be merged using any suitable techniques such as, for example, using software configured to merge variants. The variants may be merged into a multi-sample VCF file.

The candidate Mendelian violation loci 427 may be used to filter the merged variants 434 at act 428. For example, the variants that are not at the candidate Mendelian violation loci 427 may be filtered out, while variants that are at the candidate Mendelian violation loci 427 may be included in the filtered variants 429. Example filtering techniques are described herein including at least with respect to act 332 of process 320 shown in FIG. 3B.

The filtered variants 429 may be used to identify Mendelian violations 431. The Mendelian violations 431 may include one or more variants of the filtered variants 429. The variants may represent variants that are present in the genome of the child, but which are not present in the genome of either of the biological parents. Example techniques for identifying Mendelian violations are described herein including at least with respect to act 334 of process 320 shown in FIG. 3B.

In some embodiments, the Mendelian violations are filtered by read support at act 432 using aligned reads 410-1, aligned reads 410-2, and aligned reads 410-3. For example, Mendelian violations having read support that is less than a threshold value may be filtered out. Example techniques for filtering based on read support are described herein including at least with respect to act 336 of process 320 shown in FIG. 3B.

The Mendelian violations that are not filtered out at act 432 may be identified as de novo variants 413 for the child. In some embodiments, the updated plurality of variants 412 also includes inherited variants 414. The inherited variants may include variants that were included in the merged variants 434, but which were filtered out at act 428.

FIG. 4C is an illustrative example of identifying variants using joint genotyping, according to some embodiments of the technology described herein.

In the example 400, joint genotyping 442 is performed using aligned reads 410-1, aligned reads 410-2, and aligned reads 410-3. The aligned reads may include sequence reads that have been aligned to a genomic reference. For example, the aligned reads may include the sequence reads that were aligned to the family genomic reference graph at act 409 of example 400 shown in FIG. 4A. The aligned reads may be in any suitable format such as Binary Alignment Map (BAM) format or Sequence Alignment Map (SAM), as aspects of the technology described herein are not limited in this respect.

Joint genotyping, at act 442, may be performed to identify variants 443-1 for the first biological parent, variants 443-2 for the child, and variants 443-3 for the second biological parent. Example techniques for joint genotyping are described herein including at least with respect to act 362 of process 360 shown in FIG. 3C.

In the example 440, the variants identified as a result of joint genotyping at act 414 may be filtered at act 444 to obtain the updated plurality of variants 412. For example, this may include filtering variants 443-1 identified for the first biological parent, variants 443-2 identified for the child, and variants 443-3 identified for the second biological parent to obtain the updated plurality of variants 412. Example variant filtering techniques are described herein including at least with respect to act 364 of process 360 shown in FIG. 3C.

FIG. 4D is an illustrative example of aligning sequence reads to a genomic reference to identify variants, according to some embodiments of the technology described herein.

As shown in FIG. 4D, sequence reads 454 are obtained from subject 452. The subject 452 may include a member of a family trio such as, for example, a child, or either of the biological parents of the child. The sequence reads 454 may be obtained in FASTQ format. Example techniques for obtaining sequence reads from a subject are described herein including at least with respect to act 302 of process 300 shown in FIG. 3A.

In the example 450, the sequence reads 454 are aligned to a genomic reference at act 462. For example, the genomic reference may include the initial genomic reference described herein including at least with respect to act 304 of process 300 shown in FIG. 3A. The initial genomic reference graph may be a linear genomic reference or a genomic reference graph. When the initial genomic reference is a linear genomic reference, then the linear genomic reference may include reference sequence 456. For example, the reference sequence 456 may represent at least a portion (e.g., all) of a human genome. When the initial genomic reference is a genomic reference graph, then the initial genomic reference may include the reference sequence 456 augmented with pangenome variants 458. For example, the pangenome variants 458 may represent variants that are common among members of one or more specific populations (e.g., population(s) to which subject 452 belongs). Example techniques for generating a population-specific genomic reference graph are described herein including at least with respect to act 304 of process 300 shown in FIG. 3A.

Additionally, or alternatively, the genomic reference may include the family genomic reference graph described herein including at least with respect to acts 310 and 312 of process 300 shown in FIG. 3A. The family genomic reference graph may include the reference sequence 456 augmented with family variants 460 (e.g., variants 406-1, variants 406-2, and variants 406-3 shown in FIG. 4A). For example, the family variants 460 may include variants identified for subject 452. Example techniques for generating a family genomic reference graph are described herein including at least with respect to act 310 of process 300 shown in FIG. 3A and in example 470 shown in FIG. 4E.

The reference sequence 456, pangenome variants 458, and family variants 460 may each be stored in any suitable format. For example, the reference sequence 456 may be stored in FASTA format. The pangenome variants 458 and the family variants 460 may each be stored in variant call format (VCF).

In the example 450, results of aligning the sequence reads 454 to the genomic reference at act 462 may be stored in any suitable format for representing aligned sequence reads. For example, the results of the alignment may be stored in BAM format.

In some embodiments, results of aligning the sequence reads 454 to the genomic reference at act 462 are used to identify variants for the subject 452 at act 464. When the sequence reads are aligned to an initial genomic reference graph at act 464, the variants may be identified using any suitable variant calling techniques. Example variant calling techniques are described herein including at least with respect to act 306 of process 300 shown in FIG. 3A. When the sequence reads are aligned to a family genomic reference graph at act 462, the variants may be identified using any suitable variant calling techniques. Example techniques for identifying variants are described herein including at least with respect to act 314 of process 300 shown in FIG. 3A, process 320 shown in FIG. 3B, process 360 shown in FIG. 3C, example 420 shown in FIG. 4B, and example 440 shown in FIG. 4C.

In some embodiments, at act 466, the variants identified at act 464 are filtered to obtain variants 468. Example filtering techniques are described herein including at least with respect to act 336 of process 320 shown in FIG. 3B and act 364 of process 360 shown in FIG. 3C.

FIG. 4E is an illustrative example of generating a family genomic reference graph, according to some embodiments of the technology described herein. In the example 470, variants obtained for a family trio are used to generate the family genomic reference graph. The variants include variants 472-1 obtained for a first biological parent of the family trio, variants 472-2 obtained for the second biological parent of the family trio, and variants 472-3 obtained for the child of the family trio. The variants may be in any suitable format such as, for example, variant call format (VCF).

The variants may have been identified by aligning sequence reads obtained for members of the family trio to a genomic reference. For example, the variants may be initial sets of variants that were identified based on results of aligning sequence reads obtained from the members of the family trio to an initial genomic reference graph. Example techniques for identifying variants by aligning sequence reads to a genomic reference are described herein including at least with respect to acts 304-306 of process 300 shown in FIG. 3A and with respect to example 450 shown in FIG. 4D.

The variants are merged at act 478 to obtain a merged set of variants 480. For example, the variants 472-1, the variants 472-2, and the variants 472-3 are merged at act 478 to obtain the merged set of variants. The merged set of variants may be stored in variant call format.

The merged variants 480 may be used to generate the family genomic reference graph at act 484. For example, the merged set of variants may be used to augment a linear reference sequence. The linear reference sequence may represent at least a portion of (e.g., all) of a human genome. The linear reference sequence may be stored in any suitable format such as in a FASTA file, for example. Example techniques for generating a family genomic reference graph are described herein including at least with respect to act 158 of illustrative technique 150 and with respect to act 310 of process 300 shown in FIG. 3A.

### EXAMPLE

This example shows that the techniques developed by the inventors for genotyping family trios are an improvement over conventional techniques for genotyping family trios.

Experiments were performed to benchmark the performance of two embodiments of the techniques developed by the inventors for detecting de novo variants for a family trio. With respect to the first embodiment, de novo variants were identified using the techniques described herein including at least with respect to process 320 shown in FIG. 3B. With respect to the second embodiment, de novo variants were identified using the techniques described herein including at least with respect to process 360 shown in FIG. 3C. The performances of the two embodiments were compared with that of Broad Institute's Best Practices Pipeline for Germline Short Variant Discovery (BWA-GATK) and with that of GRAF Germline Variant Detection Workflow run with pan-genome graph followed by the Genomic Analysis Toolkit (GATK) GenotypeGVCFs tool (GRAF pangenome).

To evaluate performance, each technique was used to identify de novo variants for ten family trios from the Kids First data set. The de novo truth set for the ten trios (e.g., for evaluating the performance of the techniques) was prepared according to the techniques described by Richter. F, et al. (“Genomic analyses implicate noncoding de novo variants in congenital heart disease.” Nat. Genet. 52, 769-777 (2020)), which is incorporated by reference herein in its entirety.

The number of variants from the truth set that the techniques fail to detect (false negatives) was used as one of the benchmark metrics because the sensitivity of the variant calling directly influences sensitivity of diagnostic testing. Additionally, the number of extra variant calls not present in the truth set (false positives) was used as a benchmark metric since incorrect calls may mislead diagnostic testing, and complicate the identification of pathogenic variants.

FIG. 5A and FIG. 5B show results of benchmarking the first embodiment of the techniques developed by the inventors for identifying de novo variants for the family trio: HG002 (child), HG004 (mother), and HG003 (father) of the Genome in a Bottle dataset. As shown in FIG. 5A, the techniques developed by the inventors result in an increase in genotyping accuracy compared to the conventional techniques. In particular, there is an increase in precision from 20%, for the BWA-GATK techniques, to 80% for the techniques developed by the inventors. As shown in FIG. 5B, the techniques developed by the inventors result in significant decrease in spurious de novo variant calls compared to the conventional techniques. This decrease in confounding sequencing artifacts is a significant advance in the ability of computational methods to accurately identify variants acquired de novo in the child genome.

FIG. 6A-FIG. 9B show results of benchmarking the second embodiment of the techniques developed by the inventors for identifying de novo variants for the family trio.

As shown in FIG. 6A and FIG. 6B, compared to the conventional techniques, the techniques developed by the inventors bring down the number of spurious de novo variant calls (false positives), without increasing the number the number of missed de novo variant calls (false negatives). This is a significant improvement because, by reducing the number of spurious de novo variant calls, the techniques developed by the inventors significantly reduce the burden required and time wasted on evaluating non-disease-causing variants. Accordingly, the inventors have developed techniques for more efficiently and accurately identifying disease-causing de novo variants.

These results are consistent with those shown in FIGS. 7A-7B, which show the performance of each technique for identifying de novo variants for the family trio: HG002 (child), HG003 (father), and HG004 (mother) of the Genome in a Bottle (GIAB) dataset. As shown in FIG. 7A, the techniques developed by the inventors result in an increase of genotyping accuracy compared to the conventional techniques. As shown in FIG. 7B, the techniques developed by the inventors result in significant decrease in spurious de novo mutations compared to the conventional techniques.

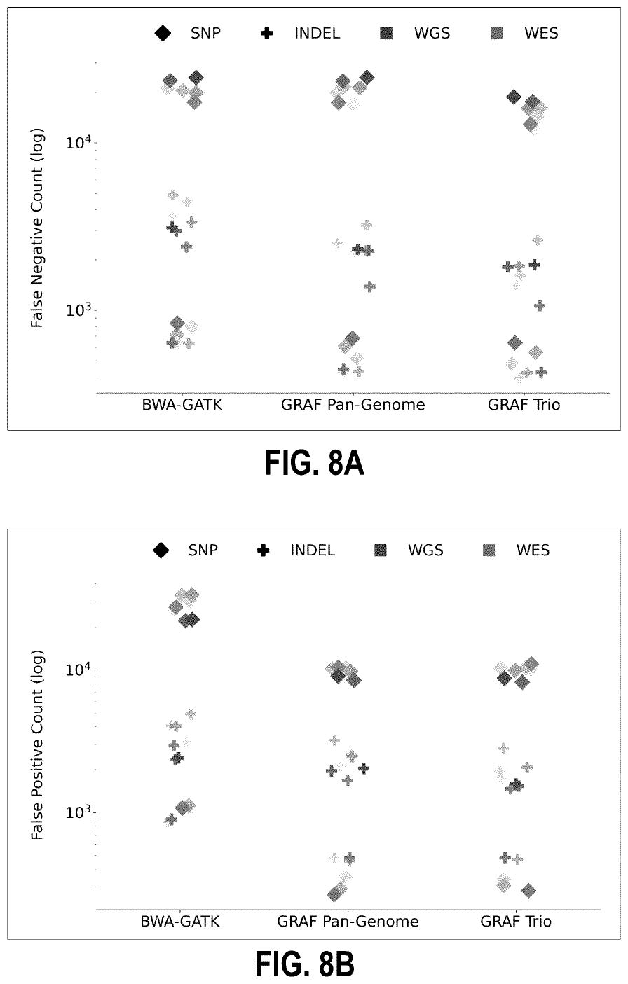

As evident from the results shown in FIGS. 8A-8B, the techniques developed by the inventors are also an improvement over conventional techniques for genotyping family trios because the use of the family genomic reference graph reduces population bias. To evaluate performance across different populations, each technique (i.e., the techniques developed by the inventors, BWA-GATK, and GRAF Pan-Genome) was used to identify de novo variants for three family trios; CEU (Northern Europeans from Utah), Ashkenazi, and Chinese, from the Genome in a Bottle (GIAB) consortium using the human reference genome GRCh38. As shown in FIGS. 8A-8B, the techniques developed by the inventors resulted in the lowest number of false negatives, meaning they enable more sensitive detection of de novo variants compared to the conventional techniques, especially for insertion deletions (indels). Furthermore, for WES sequencing data, the techniques developed by the inventors result in higher accuracy in terms of both precision and accuracy.

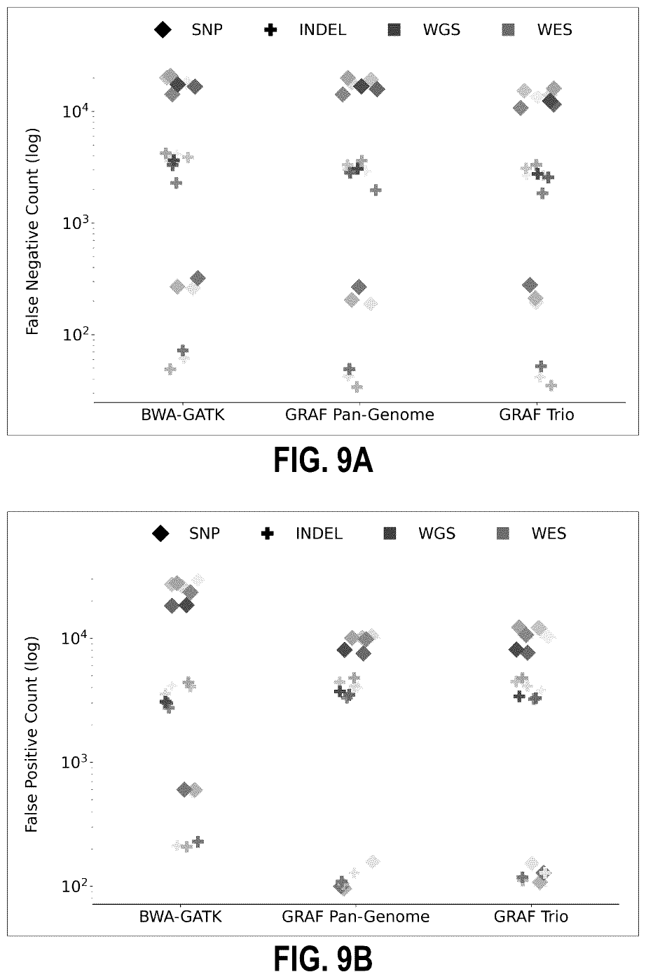

As evident from the results shown in FIGS. 9A-9B, the techniques developed by the inventors are also an improvement over the conventional techniques for identifying rare variants. To evaluate performance, each technique (i.e., the techniques developed by the inventors, BWA-GATK, and GRAF Pan-Genome) was used to identify rare variants for the GIAB datasets HG001-HG007. Rare variants were defined as variants with a minor allele frequency of less than or equal to 0.01. As shown in FIGS. 9A-9B, the techniques developed by the inventors resulted in lower numbers of missed and spurious rare variant calls. By more accurately and sensitively detecting rare variants, the techniques developed by the inventors can be used to more accurately and sensitively identify rare and complex diseases associated with those variants.

### Computer Implementation

An illustrative implementation of a computer system 1000 that may be used in connection with any of the embodiments of the technology described herein (e.g., such as the process of FIG. 2) is shown in FIG. 10. The computer system 1000 includes one or more processors 1010 and one or more articles of manufacture that comprise non-transitory computer-readable storage media (e.g., memory 1020 and one or more non-volatile storage media 1030). The processor 1010 may control writing data to and reading data from the memory 1020 and the non-volatile storage device 1030 in any suitable manner, as the aspects of the technology described herein are not limited to any particular techniques for writing or reading data. To perform any of the functionality described herein, the processor 1010 may execute one or more processor-executable instructions stored in one or more non-transitory computer-readable storage media (e.g., the memory 1020), which may serve as non-transitory computer-readable storage media storing processor-executable instructions for execution by the processor 1010.

Computing device 1000 may include a network input/output (I/O) interface 1040 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing device 1000 may also include one or more user I/O interfaces 1050, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology. CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel. It will be apparent that example aspects, as described above, may be implemented in many different forms of software, firmware, and hardware in the implementations illustrated in the figures.

### Biological Samples

Any of the methods, systems, or other claimed elements may use or be used to analyze a biological sample from a subject. The biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, or skin tissue), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, sex cord-stromal tumors, neuroendocrine tumors, gastrointestinal stromal tumors, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of a tissue, in some embodiments, refers to a sample comprising cells from a tissue. In some embodiments, the sample of the tumor comprises non-cancerous cells from a tissue. In some embodiments, the sample of the tumor comprises precancerous cells from a tissue.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue, or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, breast, and/or any type of tissue (e.g., muscle tissue, epithelial tissue, connective tissue, or nervous tissue).

Any of the biological samples described herein may be obtained from the subject using any known technique. Sec, for example, the following publications on collecting, processing, and storing biological samples, each of which are incorporated by reference herein in its entirety: Biospecimens and biorepositories: from afterthought to science by Vaught et al. (Cancer Epidemiol Biomarkers Prev. 2012 February; 21 (2): 253-5), and Biological sample collection, processing, storage and information management by Vaught and Henderson (IARC Sci Publ. 2011; (163): 23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one cell or more than one cell (i.e., a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixative. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextronse (e.g., for blood specimens). In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

### Subjects

Aspects of this disclosure relate to a biological sample that has been obtained from one or more subjects, such as one or more members of a family trio. In some embodiments, a subject is a mammal (e.g., a human, a mouse, a cat, a dog, a horse, a hamster, a cow, a pig, or other domesticated animal, a farm animal (e.g., livestock), a sport animal, a laboratory animal, a pet, and a primate). In some embodiments, a subject is a human. In some embodiments, a subject is an adult human (e.g., of 18 years of age or older). In some embodiments, a subject is a child (e.g., less than 18 years of age).

### Sequencing Data

Aspects of the disclosure may be implemented using sequencing data. For example, aspects of the disclosure relate to methods for genotyping a family trio by constructing a family genomic reference graph and analyzing sequencing data, such as sequence reads, from members of the family trio using the family genomic reference graph.

In some embodiments, sequencing data may be generated using a nucleic acid from a sample from a subject. In some embodiments, the sequencing data may indicate a nucleotide sequence of DNA from a previously obtained biological sample of a subject having, suspected of having, or at risk of having a disease. In some embodiments, the nucleic acid is deoxyribonucleic acid (DNA). In some embodiments, the nucleic acid is prepared such that the whole genome is present in the nucleic acid. When nucleic acids are prepared such that the whole genome is sequenced, it is referred to as whole genome sequencing (WGS). In some embodiment, the nucleic acid is prepared such that fragmented DNA is present in the nucleic acid. In some embodiments, the nucleic acid is processed such that only the protein coding regions of the genome remain (e.g., exomes). When nucleic acids are prepared such that only the exomes are sequenced, it is referred to as whole exome sequencing (WES). A variety of methods are known in the art to isolate the exomes for sequencing, for example, solution-based isolation wherein tagged probes are used to hybridize the targeted regions (e.g., exomes) which can then be further separated from the other regions (e.g., unbound oligonucleotides). These tagged fragments can then be prepared and sequenced.

In some embodiments, the sequencing data may include DNA sequencing data, DNA exome sequencing data (e.g., from whole exome sequencing (WES)), DNA genome sequencing data (e.g., from whole genome sequencing (WGS), shallow whole genome sequencing (sWGS), etc.), gene sequencing data, or any other suitable type of sequencing data comprising data obtained from a sequencing platform and/or comprising data derived from data obtained from a sequencing platform.

DNA sequencing data, in some embodiments, includes DNA sequence reads and/or information derived from DNA sequence reads. A DNA sequence read refers to an inferred sequence of base pairs corresponding to all or part of a DNA fragment.

DNA sequencing data, in some embodiments, includes data obtained by processing a biological sample (e.g., DNA (e.g., coding or non-coding genomic DNA) present in a biological sample) using a sequencing apparatus. DNA that is present in a sample may or may not be transcribed, but it may be sequenced using DNA sequencing platforms. Such data may be useful, in some embodiments, to determine whether the patient subject has one or more mutations associated with a particular cancer.

Sequencing data may include data generated by the nucleic acid sequencing protocol (e.g., the series of nucleotides in a nucleic acid molecule identified by any suitable generation of sequencing (Sanger sequencing, Illumina®, next-generation sequencing (NGS) etc.), as well as information contained therein (e.g., information indicative of source, tissue type, etc.) which may also be considered information that can be inferred or determined from the sequencing data.

DNA sequencing data may be acquired using any method known in the art including any known method of DNA sequencing. For example, DNA sequencing may be used to identify one or more mutations in the DNA of a subject. Any technique used in the art to sequence DNA may be used with the methods and compositions described herein. As a set of non-limiting examples, the DNA may be sequenced through single-molecule real-time sequencing, ion torrent sequencing, pyrosequencing, sequencing by synthesis, sequencing by ligation (SOLID sequencing), nanopore sequencing, or Sanger sequencing (chain termination sequencing).

In some embodiments, the sequencing data may be obtained using a sequencing platform such as a next generation sequencing platform (e.g., Illumina®, Roche®, Ion Torrent®, etc.), or any high-throughput or massively parallel sequencing platform. In some embodiments, these methods may be automated, in some embodiments, there may be manual intervention. In some embodiments, the sequencing data may be the result of non-next generation sequencing (e.g., Sanger sequencing).

In some embodiments, sequencing data comprises more than 5 kilobases (kb). In some embodiments, the size of the obtained sequencing data is at least 10 kb. In some embodiments, the size of the obtained sequencing data is at least 100 kb. In some embodiments, the size of the obtained sequencing data is at least 500 kb. In some embodiments, the size of the obtained sequencing data is at least 1 megabase (Mb). In some embodiments, the size of the obtained sequencing data is at least 10 Mb. In some embodiments, the size of the obtained sequencing data is at least 100 Mb. In some embodiments, the size of the obtained sequencing data is at least 500 Mb. In some embodiments, the size of the obtained sequencing data is at least 1 gigabase (Gb). In some embodiments, the size of the obtained sequencing data is at least 10 Gb. In some embodiments, the size of the obtained sequencing data is at least 100 Gb. In some embodiments, the size of the obtained sequencing data is at least 500 Gb.

### Example Concepts

1. A method for genotyping a family trio by constructing a family genomic reference graph and analyzing sequence reads from each member of the family trio using the family genomic reference graph, the family trio comprising a child and biological parents of the child, the method comprising: using at least one computer hardware processor to perform: obtaining the sequence reads, the sequence reads having been previously obtained by sequencing biological samples obtained from the members of the family trio; aligning the sequence reads to an initial genomic reference using at least one data structure representing the initial genomic reference; identifying, based on results of the aligning, an initial plurality of variants comprising a respective initial set of variants for each of the members of the family trio; generating the family genomic reference graph using the initial plurality of variants, the family genomic reference graph comprising nodes and edges connecting the nodes, the generating comprising generating at least one data structure storing data specifying the nodes and the edges; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, an updated plurality of variants comprising a respective updated set of variants for each of the members of the family trio.
2. The method of concept 1, further comprising: identifying, from among the updated plurality of variants, one or more de novo variants.
3. The method of concept 2, wherein identifying the one or more de novo variants comprises: identifying, based on results of aligning the at least some of the sequence reads to the family genomic reference graph, one or more variants that are detected in sequence reads obtained from a biological sample of the child and are not detected in sequence reads obtained from biological samples obtained from the biological parents of the child.
4. The method of concept 2 or 3, further comprising: identifying a disease associated with the one or more de novo variants.
5. The method of concept 1 or any other preceding concept, further comprising: identifying a plurality of variants based on the results of aligning the at least some of the sequence reads to the family genomic reference graph; and filtering the plurality of variants to obtain the updated plurality of variants, the filtering comprising for each particular variant of at least some of the plurality of variants: determining a coverage for the particular variant; and including the particular variant in the updated plurality of variants when the coverage is greater than a threshold coverage.
6. The method of concept 1 or any other preceding concept, further comprising: identifying a plurality of variants based on the results of aligning the at least some of the sequence reads to the family genomic reference graph; and filtering the plurality of variants to obtain the updated plurality of variants, the filtering comprising for each particular variant of at least some of the plurality of variants: determining a confidence that a particular variant is present in a genome of the child and genomes of the biological parents of the child; and including the particular variant in the updated plurality of variants when the confidence exceeds a threshold confidence.
7. The method of concept 1, wherein the sequence reads include first sequence reads previously obtained by sequencing a first biological sample from a first biological parent of the child, second sequence reads previously obtained by sequencing a second biological sample from a second biological parent of the child, and third sequence reads previously obtained by sequencing a third biological sample from the child, wherein aligning the sequence reads to the initial genomic reference comprises aligning the first sequence reads, the second sequence reads, and the third sequence reads to the initial genomic reference, and wherein identifying the initial plurality of variants comprises: identifying a first initial set of variants for the first biological parent based on results of aligning the first sequence reads to the initial genomic reference, identifying a second initial set of variants for the second biological parent based on results of aligning the second sequence reads to the initial genomic reference, and identifying a third initial set of variants for child based on results of aligning the third sequence reads to the initial genomic reference.
8. The method of concept 7, wherein aligning the at least some of the sequence reads to the family genomic reference graph comprises aligning, to the family genomic reference graph, at least some of the first sequence reads, at least some of the second sequence reads, and at least some of the third sequence reads.
9. The method of concept 8, wherein identifying the updated plurality of variants comprises: identifying, based on results of aligning the at least some of the first sequence reads to the family genomic reference graph, a first updated set of variants associated with the first biological parent; identifying, based on results of aligning the at least some of the second sequence reads to the family genomic reference graph, a second updated set of variants associated with the second biological parent; and identifying, based on results of aligning the at least some of the third sequence reads to the family genomic reference graph, a third updated set of variants associated with the child.
10. The method of concept 1 or any other preceding concept, wherein identifying the updated plurality of variants comprises: identifying an intermediate plurality of variants based on the results of aligning the at least some of the sequence reads to the family genomic reference graph; identifying one or more Mendelian violations using the identified intermediate plurality of variants; and filtering the one or more Mendelian violations to identify the updated plurality of variants.
11. The concept of concept 10, wherein the intermediate plurality of variants includes a first intermediate set of variants for the first biological parent, a second intermediate set of variants for the child, and a third intermediate set of variants for the second biological parent, and wherein identifying the one or more Mendelian violations comprises: identifying first differences between haplotypes of the child and haplotypes of the first biological parent using the first intermediate set of variants and the third intermediate set of variants; identifying second differences between haplotypes of the child and haplotypes of the second biological parent using the second intermediate set of variants and the third intermediate set of variants; identifying one or more Mendelian violation loci based on the first differences and the second differences; and identifying the one or more Mendelian violations using the intermediate plurality of variants and the one or more Mendelian violation loci.
12. The method of concept 1 or any other preceding concept, wherein identifying the updated plurality of variants comprises: joint genotyping the members of the family trio using the results of aligning the at least some of the sequence reads to the family genomic reference graph.
13. The method of concept 1 or any other preceding concept, wherein generating the family genomic reference graph comprises: obtaining a linear genomic reference; and augmenting the linear genomic reference with variants in the initial set of variants for each of the members of the family trio.
14. The method of concept 13, wherein augmenting the linear genomic reference comprises representing the linear genomic reference as a graph having nodes and edges and augmenting the graph with one or more nodes and one or more edges representing at least some of the initial set of variants for each of the members of the family trio.
15. The method of concept 1 or any other preceding concept, wherein the family genomic reference graph represents at least a portion of a human genome.
16. The method of concept 15, wherein the family genomic reference graph represents at least a chromosome of the human genome.
17. The method of concept 1 or any other preceding concept, wherein the family genomic reference graph represents at least 10,000,000 nucleotides, at least 50,000,000 nucleotides, at least 100,000,000 nucleotides, at least 150,000,000 nucleotides, at least 200,000,000 nucleotides, or at least 250,000,000 nucleotides.
18. The method of concept 1, wherein the family genomic reference graph is a directed acyclic graph (DAG).
19. The method of concept 1 or any other preceding concept, wherein the nodes and edges are encoded using elements in the at least one data structure, the nodes representing nucleotide sequences stored as respective strings of one or more symbols, and the edges including an edge representing a connection between at least two of the nodes.
20. The method of concept 1 or any other preceding concept, wherein the at least one data structure comprises objects representing nodes and pointers representing edges, the objects comprising a first object representing a first node of the nodes, the first object storing at least one pointer representing at least one edge in the family genomic reference graph from the first node to one or more other nodes.
21. The method of concept 1 or any other preceding concept, wherein the family genomic reference graph represents genomic information consisting of genomic information from the first parent, genomic information from the second parent, genomic information from the child, and genomic information represented by at least a portion of a linear genomic reference.
22. The method of concept 1 or any other preceding concept, wherein aligning the sequence reads to the initial genomic reference comprises aligning the sequence reads to a population-specific genomic reference.
23. The method of concept 1, wherein the population-specific genomic reference comprises a population-specific genomic reference graph representing a linear reference sequence and population-specific variants relative to the linear reference sequence.
24. A method for genotyping a family trio by using a family genomic reference graph and analyzing sequence reads from each member of the family trio using the family genomic reference graph, the family trio comprising a child and biological parents of the child, the method comprising: obtaining the sequence reads, the sequence reads having been previously obtained by sequencing biological samples obtained from each of the members of the family trio; obtaining at least one data structure storing data specifying nodes and edges of a family genomic reference graph, the family genomic reference graph having been previously generated; aligning at least some of the sequence reads to the family genomic reference graph using the at least one data structure storing data specifying the nodes and the edges of the family genomic reference graph; and identifying, based on results of aligning the at least some of the sequence reads to the family genomic reference graph, a set of variants for each of the members of the family trio.
25. A system, comprising: at least one computer hardware processor; and at least one non-transitory computer-readable storage medium storing processor-executable instructions that, when executed by the at least one computer hardware processor, causes the at least one computer hardware processor to perform the method of any one of concepts 1-24.
26. At least one non-transitory computer-readable storage medium storing processor-executable instructions that, when executed by at least one computer hardware processor, causes the at least one computer hardware processor to perform the method of any one of concepts 1-24.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an.” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for genotyping a family trio by constructing a family genomic reference graph and analyzing sequence reads from each member of the family trio using the family genomic reference graph, the family trio comprising a child and biological parents of the child, the method comprising:
using at least one computer hardware processor to perform:
obtaining the sequence reads, the sequence reads having been previously obtained by sequencing biological samples obtained from the members of the family trio;
aligning the sequence reads to an initial genomic reference using at least one data structure representing the initial genomic reference;
identifying, based on results of the aligning, an initial plurality of variants comprising a respective initial set of variants for each of the members of the family trio;
generating the family genomic reference graph using the initial plurality of variants, the family genomic reference graph comprising nodes and edges connecting the nodes, the generating comprising generating at least one data structure storing data specifying the nodes and the edges;
aligning at least some of the sequence reads to the family genomic reference graph using the at least one data structure storing data specifying the nodes and the edges of the family genomic reference graph; and
identifying, based on results of aligning the at least some of the sequence reads to the family genomic reference graph, an updated plurality of variants comprising a respective updated set of variants for each of the members of the family trio.

2. The method of claim 1, further comprising:
identifying, from among the updated plurality of variants, one or more de novo variants.

3. The method of claim 2, wherein identifying the one or more de novo variants comprises:
identifying, based on results of aligning the at least some of the sequence reads to the family genomic reference graph, one or more variants that are detected in sequence reads obtained from a biological sample of the child and are not detected in sequence reads obtained from biological samples obtained from the biological parents of the child.

4. The method of claim 2, further comprising:
identifying a disease associated with the one or more de novo variants.

5. The method of claim 1, further comprising:
identifying a plurality of variants based on the results of aligning the at least some of the sequence reads to the family genomic reference graph; and
filtering the plurality of variants to obtain the updated plurality of variants, the filtering comprising for each particular variant of at least some of the plurality of variants:
determining a coverage for the particular variant; and
including the particular variant in the updated plurality of variants when the coverage is greater than a threshold coverage.

6. The method of claim 1, further comprising:
identifying a plurality of variants based on the results of aligning the at least some of the sequence reads to the family genomic reference graph; and
filtering the plurality of variants to obtain the updated plurality of variants, the filtering comprising for each particular variant of at least some of the plurality of variants:
determining a confidence that a particular variant is present in a genome of the child and genomes of the biological parents of the child; and
including the particular variant in the updated plurality of variants when the confidence exceeds a threshold confidence.

7. The method of claim 1,
wherein the sequence reads include first sequence reads previously obtained by sequencing a first biological sample from a first biological parent of the child, second sequence reads previously obtained by sequencing a second biological sample from a second biological parent of the child, and third sequence reads previously obtained by sequencing a third biological sample from the child,
wherein aligning the sequence reads to the initial genomic reference comprises aligning the first sequence reads, the second sequence reads, and the third sequence reads to the initial genomic reference, and
wherein identifying the initial plurality of variants comprises:
identifying a first initial set of variants for the first biological parent based on results of aligning the first sequence reads to the initial genomic reference,
identifying a second initial set of variants for the second biological parent based on results of aligning the second sequence reads to the initial genomic reference, and
identifying a third initial set of variants for child based on results of aligning the third sequence reads to the initial genomic reference.

8. The method of claim 7,
wherein aligning the at least some of the sequence reads to the family genomic reference graph comprises aligning, to the family genomic reference graph, at least some of the first sequence reads, at least some of the second sequence reads, and at least some of the third sequence reads.

9. The method of claim 8, wherein identifying the updated plurality of variants comprises:
identifying, based on results of aligning the at least some of the first sequence reads to the family genomic reference graph, a first updated set of variants associated with the first biological parent;
identifying, based on results of aligning the at least some of the second sequence reads to the family genomic reference graph, a second updated set of variants associated with the second biological parent; and
identifying, based on results of aligning the at least some of the third sequence reads to the family genomic reference graph, a third updated set of variants associated with the child.

10. The method of claim 1, wherein identifying the updated plurality of variants comprises:
identifying an intermediate plurality of variants based on the results of aligning the at least some of the sequence reads to the family genomic reference graph;
identifying one or more Mendelian violations using the identified intermediate plurality of variants; and
filtering the one or more Mendelian violations to identify the updated plurality of variants.

11. The method of claim 10,
wherein the biological parents of the child include a first biological parent and a second biological parent,
wherein the intermediate plurality of variants includes a first intermediate set of variants for the first biological parent, a second intermediate set of variants for the child, and a third intermediate set of variants for the second biological parent, and
wherein identifying the one or more Mendelian violations comprises:
identifying first differences between haplotypes of the child and haplotypes of the first biological parent using the first intermediate set of variants and the third intermediate set of variants;
identifying second differences between haplotypes of the child and haplotypes of the second biological parent using the second intermediate set of variants and the third intermediate set of variants;
identifying one or more Mendelian violation loci based on the first differences and the second differences; and
identifying the one or more Mendelian violations using the intermediate plurality of variants and the one or more Mendelian violation loci.

12. The method of claim 1, wherein identifying the updated plurality of variants comprises:
joint genotyping the members of the family trio using the results of aligning the at least some of the sequence reads to the family genomic reference graph.

13. The method of claim 1, wherein generating the family genomic reference graph comprises:
obtaining a linear genomic reference; and
augmenting the linear genomic reference with variants in the initial set of variants for each of the members of the family trio.

14. The method of claim 13, wherein augmenting the linear genomic reference comprises representing the linear genomic reference as a graph having nodes and edges and augmenting the graph with one or more nodes and one or more edges representing at least some of the initial set of variants for each of the members of the family trio.

15. The method of claim 1, wherein the family genomic reference graph represents at least a chromosome of a human genome.

16. The method of claim 1, wherein the family genomic reference graph represents at least 10,000,000 nucleotides, at least 50,000,000 nucleotides, at least 100,000,000 nucleotides, at least 150,000,000 nucleotides, at least 200,000,000 nucleotides, or at least 250,000,000 nucleotides.

17. The method of claim 1,
wherein the family genomic reference graph is a directed acyclic graph (DAG),
wherein the nodes and edges are encoded using elements in the at least one data structure, the nodes representing nucleotide sequences stored as respective strings of one or more symbols, and the edges including an edge representing a connection between at least two of the nodes.

18. The method of claim 1, wherein aligning the sequence reads to the initial genomic reference comprises aligning the sequence reads to a population-specific genomic reference.

19. A system, comprising:
at least one computer hardware processor; and
at least one non-transitory computer-readable storage medium storing processor-executable instructions that, when executed by the at least one computer hardware processor, causes the at least one computer hardware processor to perform a method for genotyping a family trio by constructing a family genomic reference graph and analyzing sequence reads from each member of the family trio using the family genomic reference graph, the family trio comprising a child and biological parents of the child, the method comprising:
obtaining the sequence reads, the sequence reads having been previously obtained by sequencing biological samples obtained from the members of the family trio;
aligning the sequence reads to an initial genomic reference using at least one data structure representing the initial genomic reference;
identifying, based on results of the aligning, an initial plurality of variants comprising a respective initial set of variants for each of the members of the family trio;
generating the family genomic reference graph using the initial plurality of variants, the family genomic reference graph comprising nodes and edges connecting the nodes, the generating comprising generating at least one data structure storing data specifying the nodes and the edges;
aligning at least some of the sequence reads to the family genomic reference graph using the at least one data structure storing data specifying the nodes and the edges of the family genomic reference graph; and
identifying, based on results of aligning the at least some of the sequence reads to the family genomic reference graph, an updated plurality of variants comprising a respective updated set of variants for each of the members of the family trio.

20. At least one non-transitory computer-readable storage medium storing processor-executable instructions that, when executed by at least one computer hardware processor, causes the at least one computer hardware processor to perform a method for genotyping a family trio by constructing a family genomic reference graph and analyzing sequence reads from each member of the family trio using the family genomic reference graph, the family trio comprising a child and biological parents of the child, the method comprising:
obtaining the sequence reads, the sequence reads having been previously obtained by sequencing biological samples obtained from the members of the family trio;
aligning the sequence reads to an initial genomic reference using at least one data structure representing the initial genomic reference;
identifying, based on results of the aligning, an initial plurality of variants comprising a respective initial set of variants for each of the members of the family trio;
generating the family genomic reference graph using the initial plurality of variants, the family genomic reference graph comprising nodes and edges connecting the nodes, the generating comprising generating at least one data structure storing data specifying the nodes and the edges;
aligning at least some of the sequence reads to the family genomic reference graph using the at least one data structure storing data specifying the nodes and the edges of the family genomic reference graph; and
identifying, based on results of aligning the at least some of the sequence reads to the family genomic reference graph, an updated plurality of variants comprising a respective updated set of variants for each of the members of the family trio.
